(19) United States
(12) Patent Application Publication
(10) Pub. No.: US 2025/0134441 A1
(43) Pub. Date: May 1, 2025

(54) SYSTEMS AND PROCESSES FOR HYPERKALEMIA DETECTION USING LEAD I ECG DATA

(71) Applicant: AccurKardia, Inc., New York, NY (US)

(72) Inventors: Sluso Anne Bose, Kerala (IN); Catherine Vinnarasi Antony, Tamil Nadu (IN); Komala T.A, Karnataka (IN); Rozina Moazzam Ali Shaikh, Maharashtra (IN)

(21) Appl. No.: 18/931,521

(22) Filed: Oct. 30, 2024

Related U.S. Application Data
(60) Provisional application No. 63/594,511, filed on Oct. 31, 2023.

(51) Int. Cl. A61B 5/346; A61B 5/00; A61B 5/20; G16H 50/20

(52) U.S. Cl. CPC A61B 5/346; A61B 5/201; A61B 5/7267; G16H 50/20

(57) ABSTRACT

The present disclosure provides systems and methods for detection of hyperkalemia from Lead I electrocardiogram (ECG) signals, particularly in patients with critically high potassium levels. The methods and systems of the disclosure are further demonstrated to identifying hyperkalemia across both acute kidney disease (AKD) and chronic kidney disease (CKD) patient groups, with performance varying according to serum potassium levels.

### FIELD

The present disclosure relates to systems and processes for detection of hyperkalemia. Hyperkalemia, a medical condition characterized by abnormally high levels of potassium in the blood, poses significant health risks and requires prompt diagnosis and intervention. Elevated potassium levels can disrupt cardiac electrical activity, leading to potentially life-threatening arrhythmias. Hyperkalemia has been linked to specific ECG abnormalities, including T-wave peaking, QRS prolongation, and PR shortening.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, the invention describes systems and processes for detection of hyperkalemia applying novel approaches derived from Lead I data of electrocardiogram (ECG or EKG) recordings. Lead I specifically records the electrical potential difference between two electrodes: one generally placed on the right arm of a subject (usually the right wrist) and the other generally placed on the left arm (usually the left wrist). The present disclosure contemplates systems and methods that incorporate patterns and features obtained through a plurality of Lead I ECG signals for the detection of hyperkalemia. The methods and systems of the disclosure do not require detection of potassium from a blood to identify a subject afflicted by hyperkalemia.

In some aspects, the disclosure provides a process for screening for hyperkalemia from an electrocardiogram (ECG) signal, comprising: segmenting a set of electrical data from a Lead I electrocardiogram (lead I ECG) signal into a plurality of segments, thereby providing a set of segmented Lead I ECG electrical data; normalizing a plurality of waveforms in the set of segmented Lead I ECG data by: applying a consistent time-frame to the plurality of waveforms; applying a consistent amplitude scale to the plurality of waveforms; identifying the normalized waveform by a machine learning model trained to screen the normalized waveform for identifying a hyperkalemia waveform or a non-hyperkalemia waveform; outputting a result of the screen. In some aspects, the consistent time-frame is scaled down to a plurality of datapoints, for example 962,434 parameters or a suitable number in between these 962,434 parameters. In some aspects, the plurality of datapoints are no more than 1591 voltage measurements for each 5 second segment time-frame. In some aspects, the consistent amplitude scale is normalized from a global maximum and a global minimum. In certain aspects, the model is trained to provide a binary classification of the normalized waveform as a hyperkalemia waveform or a non-hyperkalemia waveform, e.g., the model can be instructed to apply a binary cross-entropy training metric, an Adam optimizer as a training metric, and accuracy as a training metric. The model can be trained on any suitable number of subjects, and is some cases the model is trained on at least 1,000 subjects. In some instances the model is a ResNet model. In some instances, a model of the disclosure is trained to provide a tiered classification of the normalized waveform as a mild hyperkalemia waveform, moderate hyperkalemia waveform, severe hyperkalemia waveform, or a non-hyperkalemia waveform. In some aspects, the disclosure provides a system configured to execute the steps of the aforementioned process, operatively linked to an electrocardiogram (ECG) apparatus. In some aspects, the system can be operatively linked to an wrist apparatus configured for detecting a wrist-pulse Lead I ECG signal. Such systems can be operatively linked to one or more databases of electronic medical records or clinical data, or both. In some aspects, the electrocardiogram (ECG) signal is from a subject afflicted with acute kidney disease or afflicted with chronic kidney disease.

In some aspects, the disclosure provides a process for training a model for detecting hyperkalemia from an electrocardiogram (ECG) signal, comprising: inputting into a machine learning model a set of data from a database comprising a plurality of lead I electrocardiogram (lead I ECG) signals, whereby the set of data is sub-divided into two or more bins, whereby each bin is associated with a range of a serum potassium level from a subject, the set of data comprising at least two bins selected from: a first bin comprising a first subset of Lead I electrocardiogram signals indicative of a subject having a potassium level >7.5 mEq/L; a second bin comprising a second subset of Lead I electrocardiogram signals indicative of a subject having a potassium level greater than 6.5 mEq/L and less than 7.5 mEq/L; a third bin comprising a third subset of Lead I electrocardiogram signals indicative of a subject having a potassium level greater than 6.0 mEq/L and <6.5 mEq/L; a forth bin comprising a forth subset of Lead I electrocardiogram signals indicative of a subject having a potassium level greater than 5.5 mEq/L and <6.0 mEq/L; a fifth bin comprising a fifth subset of Lead I electrocardiogram signals indicative of a subject having a potassium level greater than 5.0 mEq/L and <5.5 mEq/L; a six bin whereby the normalized waveform corresponds to a normalized waveform indicative of a subject having a potassium level <5.0 mEq/L; segmenting the set from the at least two bins into a plurality of segments, thereby providing a set of segmented lead I ECG data; normalizing a plurality of waveforms in the set of segmented lead I ECG data by: applying a consistent time-frame to the plurality of waveforms; applying a consistent amplitude scale to the plurality of waveforms; instructing a machine learning model to identify a pattern in a hyperkalemia normalized waveform or a pattern in a non-hyperkalemia normalized waveform based on a sensitivity threshold or a specificity threshold. In some aspects the consistent time-frame is scaled down to a plurality of datapoints, for example 962,434 parameters or a suitable number in between these 962,434 parameters. In some aspects, the plurality of datapoints are no more than 1591 voltage measurements for each 5 second segment time-frame. In some aspects, the consistent amplitude scale is normalized from a global maximum and a global minimum. In some instances, a model of the disclosure is trained to provide a binary classification of the normalized waveform as a hyperkalemia waveform or a non-hyperkalemia waveform. In some instances, the model has a sensitivity and/or a specificity of about 90.0% or greater when providing a binary classification of hyperkalemia. In some instances the model is instructed to apply a binary cross-entropy training metric, an Adam optimizer as a training metric, and accuracy as a training metric. In some instances the model is trained on at least 1,000 subjects. The model can be trained on any suitable number of subjects, and is some cases the model is trained on at least 1,000 subjects. In some instances the model is a ResNet model. In some instances the model is trained to provide a tiered classification of the normalized waveform as a mild hyperkalemia waveform, moderate hyperkalemia waveform, severe hyperkalemia waveform, or a non-hyperkalemia waveform. In some instances, the lead I ECG signals are from a subject afflicted with acute kidney disease. In some instances, the lead I ECG signals are from a subject afflicted with chronic kidney disease.

These aspects and other features and advantages of the invention are described below in more detail.

### Terminology

As used herein, the term “hyperkalemia”, encompasses mild, moderate, and severe hyperkalemia and is defined as a serum or plasma potassium level above the upper limits of normal, usually greater than 5.0 mEq/L to 5.5 mEq/L. In instances where a binomial (“yes or no”) method is selected to screen for hyperkalemia, hyperkalemia is defined as a normalized signal that corresponds to a serum or plasma potassium level above 5.5 mEq/L.

While mild hyperkalemia is usually asymptomatic, high potassium levels may cause life-threatening cardiac arrhythmias, muscle weakness, or paralysis. Unless an alternative nomenclature is otherwise specified, as used herein, the term “mild hyperkalemia” is defined as a serum or plasma potassium level ranging from ≥5.5 mEq/L to <6.0 mEq/L. The term “moderate hyperkalemia” is defined as a serum or plasma potassium level ranging from ≥6.0 mEq/L to <7.0 mEq/L. As used herein, the term “severe hyperkalemia” is defined as a serum or plasma potassium level ≥7.0 mEq/L.

Potassium is the chemical element with the symbol “K” and atomic number 19. Throughout the application, and particularly in figure legends, it is to be understood that the symbol “K” refers to potassium.

As used herein, electrocardiogramacess of producing an electrocardiogram (ECG or EKG), a recording of the heart's electrical activity through repeated cardiac cycles.

As used herein, “ECG” means a 12-lead ECG taken from a subject while lying down. ECG terminology has two meanings for the word “lead”: 1) the cable used to connect an electrode to the ECG recorder; and 2) the electrical view of the heart obtained from any one combination of electrodes. A standard ECG uses 10 cables to obtain 12 electrical views of the heart. The different views reflect the angles at which electrodes “look” at the heart and the direction of the heart's electrical depolarization. The electrical activity detected by the electrocardiogram machine is measured in millivolts. ECG machines are calibrated so that a raw signal with an amplitude of 1 mV moves the recording stylus vertically 1 cm.

As used herein, the expression “limb leads” refers to three bipolar leads and three unipolar leads obtained from three electrodes attached to the left arm, the right arm, and the left leg, respectively.

As used herein, the “bipolar limb” refers to the potential difference between two of the three limb electrodes.

As used herein, in some instances, the term “Lead I ECG signals” or “Lead I signals” generally refer to the potential difference between electrodes in the right arm-left arm. It is specifically contemplated that the term “Lead I ECG signal” encompasses intermittent single-lead (Lead I) ECG measurements obtained from a wrist-worn device (“wrist-pulse Lead I ECG signal”).

As used herein, the term “Lead II ECG signals” or “Lead II signals” refers to the potential difference between electrodes in the right arm-left leg.

As used herein, the term “Lead III ECG signals” or “Lead III signals” refers to the potential difference between electrodes in the left leg-left arm.

As used herein, the term “P wave” is a small deflection wave that represents atrial depolarization.

As used herein, the term “PR interval” is the time between the first deflection of the P wave and the first deflection of the QRS complex.

As used herein, the term “QRS wave complex” refers to three waves of the QRS complex representing ventricular depolarization. If a wave immediately after the P wave is an upward deflection, it is an R wave; if it is a downward deflection, it is a Q wave. Small Q waves correspond to depolarization of the interventricular septum. Q waves can also relate to breathing and are generally small and thin. They can also signal an old myocardial infarction (in which case they are big and wide). The R wave reflects depolarization of the main mass of the ventricles-hence it is frequently the largest wave. The S wave signifies the final depolarization of the ventricles, at the base of the heart.

As used herein, the term “ST segment” or “ST interval”, is the time between the end of the QRS complex and the start of the T wave. It reflects the period of zero potential between ventricular depolarization and repolarization.

As used herein, the term “T wave” represents ventricular repolarization (atrial repolarization is obscured by the large QRS complex).

As used herein, the term “about” and the term “approximately,” when used to modify a numeric value, indicate that deviations of up to 10% above and below the numeric value remain within the intended meaning of the recited value.

Designation of a range of values includes all integers within or defining the range, and all subranges defined by integers within the range.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### BRIEF DESCRIPTION OF THE DRAWINGS

The foregoing and other features and advantages of the present invention will be more fully understood from the following detailed description of illustrative configurations taken in conjunction with the accompanying drawings in which:

FIG. 1 is a schematic illustrating steps for pre-processing a raw ECG Lead I signal which include obtaining a raw signal, normalizing the signal, smoothing the signal, and outputting the transformed raw signal.

FIG. 2 is a schematic illustrating a high-level view of an ECG-waveform before and after smoothing. At a high enough level, both signals appear to be similar.

FIG. 3 illustrates a model architecture, ResNet Model, developed and described in the instant disclosure for hyperkalemia identification.

FIG. 4 depicts a flow chart with illustrative steps for curating the data.

FIG. 5 depicts a flow chart with illustrative steps for validation of data set creation.

FIG. 6 depicts a receiver operating characteristic (ROC) curve for test cases. The Y-axis represents the sensitivity (true positive rate). The x-axis represents the specificity (false positive rate). FIG. 6 illustrates performance at different K>7.5 (AUC=0.92).

FIG. 7 depicts a receiver operating characteristic (ROC) curve for test cases. FIG. 7 illustrates performance at different K values. The Y-axis represents the sensitivity (true positive rate). The x-axis represents the specificity (false positive rate). FIG. 7 illustrates performance at different K values, K>6.5 and K<7.5 (AUC=0.88).

FIG. 8 depicts a receiver operating characteristic (ROC) curve for test cases. FIG. 8 illustrates performance at different K values. The Y-axis represents the sensitivity (true positive rate). The x-axis represents the specificity (false positive rate). FIG. 8 illustrates performance at different K values, K>6 and K<6.5 (AUC=0.82).

FIG. 9 depicts a receiver operating characteristic (ROC) curve for test cases. FIG. 9 illustrates performance at different K values. The Y-axis represents the sensitivity (true positive rate). The x-axis represents the specificity (false positive rate). FIG. 9 illustrates performance at different K values, K>5.5 and K<6 (AUC=0.78).

FIG. 10 depicts a receiver operating characteristic (ROC) curve for test cases. FIG. 10 illustrates performance at different K values. The Y-axis represents the sensitivity (true positive rate). The x-axis represents the specificity (false positive rate). FIG. 10 illustrates performance at different K values, K>5 and K<5.5 (AUC=0.72).

FIG. 11 depicts an overlay of various operating characteristic (ROC) curve for test cases: K>7.5 (AUC=0.92), K>6.5 (AUC=0.89), K≥6.5 and ≤7.5 (AUC=0.88), K>6 and ≤6.5 (AUC=0.82), K≥5.5 and ≤6 (AUC=0.78), and K≥5 and ≤5.5 (AUC=0.72).

FIG. 12 depicts a flow chart with illustrative steps for validation of data set creation on subjects with acute kidney disease.

FIGS. 13-18 collectively depict receiver operating characteristic (ROC) curves validated on subjects with acute kidney disease. FIGS. 13-18, collectively are charts depicting performance at different K values. In FIGS. 13-18, collectively, the Y-axis represents the sensitivity (true positive rate) and the x-axis represents the specificity (false positive rate). FIG. 13 illustrates performance at different K values when K>7.5 (AUC=0.87) on subjects with acute kidney disease. FIG. 14 illustrates performance at different K values when K>6.5 and K<7.5 (AUC=0.87) on subjects with acute kidney disease. FIG. 15 illustrates performance at different K values when K>6 and K<6.5 (AUC=0.77) on subjects with acute kidney disease. FIG. 16 illustrates performance at different K values when K>5.5 and K<6 (AUC=0.70) on subjects with acute kidney disease. FIG. 17 illustrates performance at different K values when K>5 and K<5.5 (AUC=0.58) on subjects with acute kidney disease. FIG. 18 depicts an overlay of various operating characteristic (ROC) curve for test cases on patients with acute kidney disease.

FIGS. 19-24 collectively depict receiver operating characteristic (ROC) curves validated on subjects with chronic kidney disease. FIGS. 19-24, collectively are charts depicting performance at different K values. In FIGS. 19-24, collectively, the Y-axis represents the sensitivity (true positive rate) and the x-axis represents the specificity (false positive rate). FIG. 19 illustrates performance at different K values when K>7.5 (AUC=0.88) on subjects with chronic kidney disease. FIG. 20 illustrates performance at different K values when K>6.5 and K<7.5 (AUC=0.79) on subjects with chronic kidney disease. FIG. 21 illustrates performance at different K values when K>6 and K<6.5 (AUC=0.74) on subjects with chronic kidney disease. FIG. 22 illustrates performance at different K values when K>5.5 and K<6 (AUC=0.70) on subjects with chronic kidney disease. FIG. 23 illustrates performance at different K values when K>5 and K<5.5 (AUC=0.65) on subjects with chronic kidney disease. FIG. 24 depicts an overlay of various operating characteristic (ROC) curve for test cases on patients with acute kidney disease.

It should be understood that the drawings are not necessarily to scale (e.g., schematics), and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### I. Overview

All of the functionalities described in connection with one embodiment of the methods, compositions, or formulations described herein are intended to be applicable to the additional configurations of the methods, compositions, or formulations described herein except where expressly stated or where the feature or function is incompatible with the additional configurations. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

Alterations observed in electrocardiograms (ECGs) are believed to be pivotal indicators in the identification of cardiac abnormalities. Potassium, a vital electrolyte within cardiac cells, is speculated to play a significant influence on ECG readings. Dysregulation of potassium levels, encompassing conditions like hyperkalemia and hypokalemia, can precipitate cardiac dysfunction, potentially posing life-threatening risks that necessitate immediate intervention.

A substantial proportion of cardiac arrests in adults who do not have concurrent cardiac disorders can be attributed to metabolic irregularities. Potassium is a critical electrolyte that exerts an influence on the transmembrane potential within cardiac cells. The electrical stability of the heart is highly susceptible to changes in potassium concentration, with both increases and decreases having significant impacts on the electrophysiology of cardiac muscle.

ECG changes can be potentially informative for recognizing electrolyte abnormalities. It has been reported in the art that hyperkalemia may correlate with certain ECG manifestations. See, e.g., Levis J T. ECG diagnosis: hyperkalemia. Perm J. 2013; 17:69. In some cases, it has been further reported that certain ECG changes, including expansive QRS complex, peaked T-wave, prolonged QT-interval, and hidden p-wave might be associated with hyperkalemia. See, e.g., Webster A, Brady W, Morris F. Recognizing signs of danger: ECG changes resulting from an abnormal serum potassium concentration. Emerg Med J. 2002; 19:74-7. These results, however, are based on patient cohorts that are quite small and potentially merely comprise data of severe outliers. There is a significant need in the art for processes and systems that can robustly identify hyperkalemia, particularly in its early stages, e.g., from more subtle ECG patterns.

In some aspects, the invention is based on the development of novel systems, processes, and methods for detection of hyperkalemia. In some aspects, the disclosure provides systems and methods for detection of hyperkalemia from Lead I ECG signals only. Lead I ECG signals specifically record the electrical potential difference between two electrodes: one placed on the right arm (usually the right wrist) and the other on the left arm (usually the left wrist). More recent technological advances have also allowed somewhat interchangeable signals to be obtained from intermittent single-lead (Lead I) ECG measurements obtained from a wrist-worn device (“wrist-pulse Lead I ECG signal”). The disclosure describes processes that, first, segment raw ECG Lead I signals into smaller segments, e.g. 5 sec time-frames, to facilitate management of the data. Although 5 sec time-frames were first developed in embodiments described in the examples, other suitable time-frames could be utilized in alternative embodiments (e.g. 1-10 sec time-frames). Segmentation of the ECG Lead I signal into smaller time frames led to the generation of a plurality of sets of segmented ECG data. Each segment in the plurality of sets of segmented ECG data comprises a range of individual voltage signals, illustrated as waveforms. The disclosure devised a process whereby a consistent time-frame was applied to each waveform thereby scaling down the number of individual voltage signals to a consistent base line of datapoints, e.g., at most 1591 voltage measurements for each 5 second segment time-frame. A consistent amplitude scale was also applied to a range of individual voltage signals to normalize the waveforms according to a global maximum and a global minimum. Thus, a plurality of waveforms in each time segment were normalized for both its time and amplitude scales. Normalizing the time scale ensures that all segments have a consistent duration, facilitating accurate comparisons and analysis. Similarly, normalizing the amplitude scale ensures that the signal amplitudes are standardized, allowing for meaningful comparisons between different segments. Thus, a normalize waveform could be associated as a hyperkalemia waveform or a non-hyperkalemia waveform.

Examples presented herein demonstrated use of such systems and methods in successfully detecting hyperkalemia from Lead I ECG signals. In some aspects, the disclosure provides a paradigm shift from the standard techniques typically used in the art for hyperkalemia detection.

### II. Processes for Detecting Hyperkalemia

Hyperkalemia can result either from a shift of potassium out of cells or from abnormal renal potassium excretion. Typically, the distinct underlying causes of hyperkalemia are accompanied by different clinical manifestations. For instance, cell shift can lead to transient increases in the plasma potassium concentration, whereas decreased renal excretion of potassium can lead to sustained hyperkalemia. Further, impairments in renal potassium excretion can be the result of reduced sodium delivery to the distal nephron, decreased mineralocorticoid level or activity, or abnormalities in the cortical collecting duct. In some instances, all three of these, or other, perturbations can be present. Yet, detection of hyperkalemia is typically made when serum potassium concentration levels exceeds a range of 5.0 mEq/L to 5.5 mEq/L.

In some aspects the disclosure provides a process for screening for hyperkalemia from an electrocardiogram (ECG) signal, comprising: segmenting a set of electrical data from a lead I electrocardiogram (lead I ECG) signal into a plurality of segments, thereby providing a set of segmented lead I ECG electrical data; normalizing a plurality of waveforms in the set of segmented lead I ECG data by: i) applying a consistent time-frame to the plurality of waveforms; ii) applying a consistent amplitude scale to the plurality of waveforms; identifying the normalized waveform by a machine learning model trained to screen the normalized waveform for identifying a hyperkalemia waveform or a non-hyperkalemia waveform; outputting a result of the screen.

In many aspects, the consistent time-frame is scaled down to a plurality of datapoints. Typically, an ECG instrument records each lead separately, either sequentially or, in some instruments, several leads can be recorded simultaneously. As the stylus moves, depending on the voltage it is reflecting, the recording paper moves at a constant, present speed generally of 25 mm/sec. Hence time is represented on the recording paper by the horizontal axis, and voltage is reflected in the vertical axis. The signal is recorded on a grid, with lines 1 mm apart in both the vertical and horizontal axes. In the horizontal axis, each 1 mm generally represents 0.04 second (40 msec), and every 5 mm, designated by a bold line, indicates 0.2 second. The recording is generally standardized, so that 1 mm vertical deflection reflects 0.1 mV; 5 mm, again indicated by a more bold line, represents 0.5 mV. If the electrocardiogram is recorded at a different paper speed (such as twice the conventional rate) or with a voltage other than the conventional, these alterations must be recorded and taken into account when measuring the various intervals and waves of the ECG. If the electrocardiogram is recorded in a wrist-pulse device these alterations must be taken int account when measuring the various intervals.

By convention, the first upward deflection from the baseline is termed the P wave, and it reflects atrial depolarization. It is understood that in healthy scenarios, the P wave should not exceed 2.5 mm in height nor 0.11 second in width (i.e., less than three small boxes high and wide). Ventricular depolarization is represented by the QRS complex. The Q wave is the first negative deflection from the baseline after the P wave, but preceding an upward deflection. Normally, the Q wave reflects ventricular septal depolarization, and its duration does not exceed 0.03 second. The R wave is the first positive deflection after the P wave, reflecting depolarization of the ventricular mass. The S wave is the negative deflection following the positive R wave representing later ventricular depolarization. Any positive deflection following an S wave is labeled R′ (“R-prime”); any negative deflection following an R′ is labeled S′. By convention, an uppercase R or S infers a large deflection, whereas a lowercase r or s infers a smaller deflection. The T wave reflects repolarization of the ventricle and may be represented as either a positive or negative deflection following the QRS complex. The area incorporated within the T wave approximates that within the QRS complex, and its polarity is roughly the same as the principal QRS polarity. Occasionally, another wave, the U wave, may follow the T wave, and it is generally of the same polarity as the T wave. The mechanism of the U wave is unknown, though it may reflect repolarization of papillary muscles, or simply represent an afterpotential. The PR interval is the time from the beginning of the P wave to the beginning of the QRS, whether initiated by a Q or an R, and this interval indicates the time required for the atria to depolarize, and for the electrical current to conduct through the atrioventricular node and bundle branches until the ventricle depolarizes. The QRS interval is that interval from the beginning of the Q wave to the end of the S wave, incorporating ventricular depolarization. The QT interval is the time from the beginning of the Q wave to the end of the T wave, incorporating both ventricular depolarization and repolarization. The PR segment is that portion of the recording between the end of the P wave and the beginning of the QRS. The ST segment is that portion of the recording, generally represented by a horizontal line, from the end of ventricular depolarization, whether represented by an R wave or an S wave, to the beginning of the T wave. All of these waves (i.e., “waveforms”) generate a plurality of datapoints associated with a certain voltages in the Y-axis and times on the X-axis. The disclosure provides a method that first segments a set of data (e.g., electrical data or voltage data) from a lead I electrocardiogram (lead I ECG) signal into a plurality of segments defined by a consistent time-frame, thereby providing a set of segmented lead I ECG electrical data. The consistent time frame can be any reasonable amount of time ranging from milliseconds to about 10 seconds. Typically, each consistent time frame will comprise a large enough number of individual datapoints to provide sufficient information for a machine learning model to distinguish features. In some cases, the plurality of datapoints are about 1,000,000 datapoints (e.g., from about 1,000 to about 962,434 parameters) in a 5-second consistent time frame.

In some aspects the disclosure provides a process for normalizing a plurality of waveforms in the set of segmented lead I ECG data by applying a consistent time-frame to the plurality of waveforms thereby limiting the number of possible datapoints to a time-frame and normalizing the time-frame. In some cases, the plurality of datapoints in a normalized time-frame are no more than 10,000, no more than 9,500, no more than 9,000, no more than 8,500, no more than 8,000, no more than 7,500, no more than 7,000, no more than 6,500, no more than 6,000, no more than 5,500, no more than 5,000, no more than 4,500, no more than 4,000, no more than 3,500, no more than 3,000, no more than 2,500, no more than 2,000, no more than 1,500, voltage measurements for each 5 second segment time-frame. In one particular implementation of the systems and processes of the disclosure about 1,591 voltage measurements for each 5 second segment time-frame were considered to normalize each wave form. The disclosure contemplates that a commensurate number of waveforms can be applied to normalize time frames of different lengths.

In some aspects the disclosure provides a process for normalizing an amplitude scale of a plurality of waveforms in the set of segmented lead I ECG data by applying a global maximum and a global minimum. Electrocardiogramaignals are generally signals of short intervals of characteristic oscillation, characterized by wavelengths of known amplitude. An amplitude scale (or dilation) relates to how “stretched” or “squished” a waveform is. Location defines where the wave is positioned with regards to time (or space). Application of a global maximum and a global minimum provides a normalized set of data within standardized sets of segmented lead I ECG data.

In some aspects, the normalized waveform is inputted into a machine learning model trained to screen the normalized waveform for identifying a hyperkalemia waveform or a non-hyperkalemia waveform. In some aspects the model is trained to provide a binary classification (“yes or no”) of the normalized waveform as a hyperkalemia waveform or a non-hyperkalemia waveform. In some instances the model is trained to provide a tiered classification of the normalized waveform as a mild hyperkalemia waveform, moderate hyperkalemia waveform, severe hyperkalemia waveform, or a non-hyperkalemia waveform.

In some aspects the disclosure describes a process for training a model for detecting hyperkalemia from an electrocardiogram (ECG) signal, comprising: inputting into a machine learning model a set of data from a database comprising a plurality of lead I electrocardiogram (lead I ECG) signals, whereby the set of data is sub-divided into bins, whereby each bin is associated with a range of a serum potassium level from a subject, the set of data comprising at least two bins selected from: a first bin comprising a first subset of Lead I electrocardiogram signals indicative of a subject having a potassium level >7.5 mEq/L; a second bin comprising a second subset of Lead I electrocardiogram signals indicative of a subject having a potassium level ≥6.5 mEq/L and ≤7.5 mEq/L; a third bin comprising a third subset of Lead I electrocardiogram signals indicative of a subject having a potassium level >6.0 mEq/L and <6.5 mEq/L; a forth bin comprising a forth subset of Lead I electrocardiogram signals indicative of a subject having a potassium level ≥5.5 mEq/L and <6.0 mEq/L; a fifth bin comprising a fifth subset of Lead I electrocardiogram signals indicative of a subject having a potassium level ≥5.0 mEq/L and <5.5 mEq/L; a six bin whereby the normalized waveform corresponds to a normalized waveform indicative of a subject having a potassium level <5.0 mEq/L; segmenting the set from the at least two bins into a plurality of segments, thereby providing a set of segmented lead I ECG data; normalizing a plurality of waveforms in the set of segmented lead I ECG data by: applying a consistent time-frame to the plurality of waveforms; applying a consistent amplitude scale to the plurality of waveforms; instructing a machine learning model to identify a pattern in a hyperkalemia normalized waveform or a pattern in a non-hyperkalemia normalized waveform based on a sensitivity threshold or a specificity threshold. In principle, any number of bins can be used to train a model, e.g., 1, 2, 3, 4, 5, 6, 7, 8, 9, 10, 11, 12, 13, 14, 15, 16, 17, 18, 19, 20, or another suitable number. One of skill in the art will recognize that each bin can be associated with a defined potassium level, either broader or narrower than the aforementioned ranges, and the model can be refined based on the number of bins and its associated ranges. Each one of these time-frames can be associated with the plurality of datapoints described above.

In some aspects the disclosure provides a process for screening hyperkalemia from an electrocardiogram (ECG) dataset, comprising: segmenting a set of electrical data from a lead I electrocardiogram (lead I ECG) signal into a plurality of segments, thereby providing a set of segmented lead I ECG electrical data; normalizing a plurality of waveforms in the set of segmented lead I ECG data by: i) applying a consistent time-frame to the plurality of waveforms; ii) applying a consistent amplitude scale to the plurality of waveforms; screening the normalized waveform into one or more bins by matching the normalized waveforms to at least one normalized reference waveforms in a database comprising a plurality of normalized reference wavelengths, wherein the database comprises: a first bin whereby the normalized waveform corresponds to a normalized reference waveform indicative of a subject having a potassium level >7.5 mEq/L; a second bin whereby the normalized waveform corresponds to a normalized waveform indicative of a subject having a potassium level ≥6.5 mEq/L and ≤7.5 mEq/L; a third bin whereby the normalized waveform corresponds to a normalized waveform indicative of a subject having a potassium level ≥6.0 mEq/L and <6.5 mEq/L; a forth bin whereby the normalized waveform corresponds to a normalized waveform indicative of a subject having a potassium level ≥5.5 mEq/L and <6.0 mEq/L; a fifth bin whereby the normalized waveform corresponds to a normalized waveform indicative of a subject having a potassium level ≥5.0 mEq/L and <5.5 mEq/L; a six bin whereby the normalized waveform corresponds to a normalized waveform indicative of a subject having a potassium level <5.0 mEq/L; and outputting the classification result thereby screening the hyperkalemia status.

### Systems for Detecting Hyperkalemia

In some aspects, the disclosed provides a system to identify hyperkalemia, preferably a system for screening hyperkalemia according to a range of potassium levels demonstrated to be associated with a normalized ECG Lead I signal. In many instances, systems, platforms, software, networks, and methods described herein include a digital processing device, or use of the same. In further configurations, the digital processing device includes one or more hardware central processing units (CPUs), i.e., processors that carry out the device's functions, such as storing a curated database of waveforms and weights identifying hyperkalemia in the validation data sets. See, e.g., Example 1 and 2 for the development of an instant database The system disclosed herein or a computer system used in the analyses of a set of normalized waveforms, can share the results with a third-party from any other facility, such as a hospital a clinical facility or another heath care organization. In still further configurations, the digital processing device further comprises an operating system configured to perform executable instructions, such as instructions required to normalize a raw waveform from a Lead I ECG and perform steps similar or substantially similar to the steps described in the instant Examples for the validation conducted on the five subset grouped according to the serum potassium. The system of the disclosure can normalize a raw waveform as described herein, preferably Lead I ECG waveform, compare the normalize waveform to a reference normalized waveform on the instantly described database, and output the result.

In some aspects, a system described herein is optionally connected a computer network. In further configurations, the system is optionally connected to the Internet such that it accesses the World Wide Web, e.g., the reference database of a plurality of normalized waveforms can be stored in the World Wide Web, including normalized waveforms that can provide a binary (“yes or no”) identification of hyperkalemia based on a defined cut-off (e.g., 5.5 mEq/L of K). In other embodiments, it is contemplated that the reference database comprises a plurality of normalized waveforms indicative of a subject having a potassium level >7.5 mEq/L, a potassium level ≥6.5 mEq/L and ≤7.5 mEq/L, a potassium level ≥6.0 mEq/L and <6.5 mEq/L, a potassium level ≥5.5 mEq/L and <6.0 mEq/L, a potassium level ≥5.0 mEq/L and <5.5 mEq/L, or a potassium level <5.0 mEq. Once of skill in the art will understand that databases comprises variations in the K levels described above can also be suitable, e.g., for the purposes of training a model for detecting hyperkalemia and supporting detection of hyperkalemia. In still further configurations, the system is optionally connected to a cloud computing infrastructure. In other configurations, the digital processing device is optionally connected to an intranet. In other configurations, the digital processing device is optionally connected to a data storage device. In other configurations, the digital processing device could be deployed on premise or remotely deployed in the cloud. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable system that can either be functionally connected with electrocardiogram ECG or EKG equipment, either directly or via a third-party, for on-site monitoring and hyperkalamia detection classification outputting. Such hyperkalemia classification systems of the disclosure provide a range of potential serum potassium levels that can be associated with an ECG within a certain confidence interval without necessarily requiring a contemporaneously potassium test.

In some aspects, a system of the disclosure includes an operating system configured to perform executable instructions, e.g., associate a raw ECG waveform into one or more hyperkalemia “bins” or categories listing potassium ranges within certain confidence intervals. The operating system is, for example, software, including programs and data, which manages the overall system's hardware and provides services for execution of applications. Those of skill in the art will recognize that suitable server operating systems include, by way of non-limiting examples, FreeBSD, OpenBSD, NetBSD, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some aspects, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. In the specific Examples provided herein, the data was analyzed using IBM SPSS version 24.

In some aspects, a hyperkalemia detection system of the disclosure includes a storage and/or memory device. The storage and/or memory device can be one or more physical apparatuses used to store data or programs on a temporary or permanent basis. In some configurations, the device is volatile memory and requires power to maintain stored information. In some configurations, the device is non-volatile memory and retains stored information when the digital processing device is not powered. In further configurations, the non-volatile memory comprises flash memory. In some configurations, the non-volatile memory comprises dynamic random-access memory (DRAM). In some configurations, the non-volatile memory comprises ferroelectric random access memory (FRAM). In some configurations, the non-volatile memory comprises phase-change random access memory (PRAM). In other configurations, the device is a storage device including, by way of non-limiting examples, CD-ROMs, DVDs, flash memory devices, magnetic disk drives, magnetic tapes drives, optical disk drives, and cloud computing based storage. In further configurations, the storage and/or memory device is a combination of devices such as those disclosed herein.

In some configurations, a hyperkalemia detection system of the disclosure includes a display to send visual information to a third-party, such as health care facility, a physicians office, or a relative of the subject being monitored for hyperkalemia or undertaking an ECG. In some configurations, the display is a cathode ray tube (CRT). In some configurations, the display is a liquid crystal display (LCD). In further configurations, the display is a thin film transistor liquid crystal display (TFT-LCD). In some configurations, the display is an organic light emitting diode (OLED) display. In various further configurations, on OLED display is a passive-matrix OLED (PMOLED) or active-matrix OLED (AMOLED) display. In some configurations, the display is a plasma display. In other configurations, the display is a video projector. In still further configurations, the display is a combination of devices such as those disclosed herein. In certain configurations the performance of the deep neural network (DNN) in predicting hyperkalemia through ECG using lead-1 is outputted on the display, alongside its respective sensitivity or specificity for that range.

In some configurations, a hyperkalemia detection system includes an input device to receive information from a user. In some configurations, the input device is an electrocardiogram machine, and the hyperkalemia detection system is formatted to receive a plurality of data from electrical activity of the heart as be measured on the surface of the skin, i.e., 12 lead ECG or a wrist-pulse ECG signal. This includes electrocardiogram data from one or more of: 1) RA electrode, placed on the right arm; 2) LA electrode, place on the left arm; 3) RL electrode, placed on the right leg; 4) LL electrode placed on the left leg; 5) V₁ electrode, placed in the fourth intercostal space (between ribs 4 and 5); 6) V₂ electrode, placed in the fourth intercostal space (between ribs 4 and 5); 7) V₃ electrode, placed between leads V₂ and V₄; 8) V₄ electrode, placed in the fifth intercostal space (between ribs 5 and 6); 9) V₅ electrode, placed horizontally even with V₄; and 10) V₆ electrode, placed horizontally even with V₄ and V₅ in the mid-axillary line. In still further configurations, the input device is a combination of devices such as those disclosed herein. In still further configurations, the input device detects wrist-pulse ECG signal data.

In some configurations, a hyperkalemia detection system includes a digital camera. In some configurations, a digital camera captures digital images, such as, e.g., a schematic representation of a ECG. In some configurations, a digital camera captures still images of the ECG for further analysis by the system, and the system is able to segment the signal.

### Non-Transitory Computer Readable Storage Medium

In many aspects, the processes and systems that provide the hyperkalemia detection system disclosed herein include one or more non-transitory computer readable storage media encoded with a program including instructions executable by the operating system of an optionally networked digital processing device. For instance, in some aspects, the processes of the disclosure comprise creating data files associated with a plurality of Lead I ECG waveforms from a set of data. In certain configurations, the system of the disclosure incorporates a database of normalized waveforms that can be used as a reference. In other configurations, a database of the disclosure may not require a reference database. The non-transitory computer storage medium can store data files associated with one or more 12-lead ECG measurements described herein.

Further the processes and systems that provide the hyperkalemia detection system disclosed herein can include one or more non-transitory computer readable storage media encoded with a program including instructions executable by the operating system of an optionally networked digital processing device configured to create data files associated with a plurality of waveforms (including raw ECG waveforms from one or more 12-lead ECG measurements, raw Lead I ECG waveforms, and normalized ECG Lead I waveforms). In preferred configurations, the data is further analyzed by a process of the disclosure that groups the results into one of five combinations (or “bins”) correlating to waveforms from a subject having a potassium level >7.5 mEq/L, a potassium level ≥6.5 mEq/L and ≤7.5 mEq/L, a potassium level ≥6.0 mEq/L and <6.5 mEq/L, a potassium level ≥5.5 mEq/L and <6.0 mEq/L, a potassium level ≥5.0 mEq/L and <5.5 mEq/L, or a potassium level <5.0 mEq. In combination with the analysis of the waveform, the output of the process described herein can provide a differential classification of hyperkalemia. The non-transitory computer storage medium can store data files associated with all of the hyperkalemia classifications described herein.

In further configurations, a computer readable storage medium is a tangible component of a system of the disclosure. In still further configurations, a computer readable storage medium is optionally removable from a digital processing device. In some configurations, a computer readable storage medium includes, by way of non-limiting examples, CD-ROMs, DVDs, flash memory devices, solid state memory, magnetic disk drives, magnetic tape drives, optical disk drives, storage area network (SAN), cloud computing systems and services, and the like. In some cases, the program and instructions are permanently, substantially permanently, semi-permanently, or non-transitorily encoded on the media. Such computer readable storage medium is also suitable for storing the set of data contemplated by the disclosure.

### Computer Program

The processes and systems that provide the hyperkalemia detection system disclosed herein typically include at least one computer program. A computer program includes a sequence of instructions, executable in the digital processing device's CPU, written to perform a specified task. Example 1 provides exemplary steps used by Applicants to, e.g., smooth a waveform (i.e., Savitzky-Golay (S-G) filter). One of skill in the art will understand that other filters may be suitable. The S-G filter, for instance, is a digital filter that effectively reduces noise and unwanted variations in the signal while preserving the underlying prominent features.

In light of the disclosure provided herein, those of skill in the art will recognize that a computer program may be written in various versions of various languages. In some configurations, a computer program comprises one sequence of instructions. For instance, a program may be written to achieve the same sequence of instructions or substantially the same sequence of instructions instantly described to develop a hyperkalemia detection model. For instance, in developing the model, the disclosure contemplates systems having computer programs configured to 1) quantify the disparity between the model's predictions and the true binary labels, guiding the optimization process to minimize this discrepancy (e.g., loss=‘binary_crossentropy’); 2) dynamically updating the model's weights during training (e.g., optimizer=‘adam’: The “optimizer”); 3) measuring the ratio of correctly predicted instances to the total instances (e.g., metrics=[‘accuracy’]); 4) dynamically monitor plateaus during training (e.g., reduce_lr=ReduceLROnPlateau( )); and 5) to prevent overfitting (e.g., early_stopping=EarlyStopping(patience=50, min_delta=0.0001). A program may be written to achieve the same sequence of instructions or substantially the same sequence of instructions instantly described to apply a hyperkalemia detection system to the classification of a raw Lead I ECG signal. For instance, the system may be instructed to normalize the Lead I ECG signal, to classify the normalized signal into a hyperkalemia “bin” according to the normalized signal, and the system may be instructed to output the results. In some configurations, a computer program comprises a plurality of sequences of instructions. In some configurations, a computer program instruction is provided from one location (e.g., a computer program that is functionally connected to an ECG apparatus or another medical apparatus). In other configurations, a computer program is provided from a plurality of locations (e.g., ECG signal is provide to a third-party and the analysis occurs in yet another site). In various configurations, a computer program includes one or more software modules. In various configurations, a computer program includes, in part or in whole, one or more web applications, one or more mobile applications, one or more standalone applications, one or more web browser plug-ins, extensions, add-ins, or add-ons, or combinations thereof.

### Web Application

In some configurations, the processes and systems that provide the hyperkalemia detection system disclosed herein include a web application. In light of the disclosure provided herein, those of skill in the art will recognize that a web application, in various configurations, utilizes one or more software frameworks and one or more database systems. In some configurations, a web application is created upon a software framework such as Microsoft®.NET or Ruby on Rails (RoR). In some configurations, a web application utilizes one or more database systems including, by way of non-limiting examples, relational, non-relational, object oriented, associative, and XML database systems. In further configurations, suitable relational database systems include, by way of non-limiting examples, Microsoft® SQL Server, mySQL™, and Oracle®. Those of skill in the art will also recognize that a web application, in various configurations, is written in one or more versions of one or more languages. A web application may be written in one or more markup languages, presentation definition languages, client-side scripting languages, server-side coding languages, database query languages, or combinations thereof. In some configurations, a web application is written to some extent in a markup language such as Hypertext Markup Language (HTML), Extensible Hypertext Markup Language (XHTML), or extensible Markup Language (XML). In some configurations, a web application is written to some extent in a presentation definition language such as Cascading Style Sheets (CSS). In some configurations, a web application is written to some extent in a client-side scripting language such as Asynchronous Javascript and XML (AJAX), Flash® Actionscript, Javascript, or Silverlight®. In some configurations, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some configurations, a web application is written to some extent in a database query language such as Structured Query Language (SQL). In some configurations, a web application integrates enterprise server products such as IBM® Lotus Domino®. A web application for providing a career development network for artists that allows artists to upload information and media files, in some configurations, includes a media player element. In various further configurations, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some configurations, the systems that provide the hyperkalemia detection system disclosed herein include a mobile application provided to a mobile digital processing device. In some configurations, the mobile application is provided to a mobile digital processing device at the time it is manufactured. In other configurations, the mobile application is provided to a mobile digital processing device via the computer network described herein. It is specifically contemplated that the hyperkalemia detection system is configured for display on a mobile device. In specific instances, the hyperkalemia detection system outputs a classification result for display on an interface, e.g., graphical user interface. In certain configurations, the hyperkalemia classification output comprises one or a combination of two or more of text, color, imagery, or sound to alert the subject or the technician, physician, or the like administering the ECG of a subject. Specifically, the outputting operation can send an alert to an end-user if the results of the classification are “mild hyperkalemia”, “moderate hyperkalemia”, “severe hyperkalemia”, and indicative of a poor outcome.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some configurations, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some configurations, a computer program includes one or more executable complied applications.

### Software Modules

The processes and systems that provide the hyperkalemia detection system disclosed herein include, in various configurations, software, server, and database modules. A specific database contemplated by the disclosure is described in the Examples. In view of the disclosure provided herein, software modules are created by techniques known to those of skill in the art using machines, software, and languages known to the art. The software modules disclosed herein are implemented in a multitude of ways. In various configurations, a software module comprises a file, a section of code, a programming object, a programming structure, or combinations thereof. In further various configurations, a software module comprises a plurality of files, a plurality of sections of code, a plurality of programming objects, a plurality of programming structures, or combinations thereof. In various configurations, the one or more software modules comprise, by way of non-limiting examples, a web application, a mobile application, and a standalone application. In some configurations, software modules are in one computer program or application. In other configurations, software modules are in more than one computer program or application. In some configurations, software modules are hosted on one machine. In other configurations, software modules are hosted on more than one machine. In further configurations, software modules are hosted on cloud computing platforms. In some configurations, software modules are hosted on one or more machines in one location. In other configurations, software modules are hosted on one or more machines in more than one location.

### EXAMPLES

### Example 1—Model Development for Hyperkalemia Detection

This example details the development of a robust model for hyperkalemia detection:

Database Development from 10 Million Patients

The instant model was developed as part of the Mayo Clinic Platform Accelerate Cohort 3 program. As part of this program, Applicant analyzed a dataset encompassing de-identified electrocardiograms (ECGs) from a cohort of 10 million patients. Each ECG dataset in the cohort was characterized by 8 distinct waveforms, aligning with the 8 different leads of a traditional ECG: I, II, V1, V2, V3, V4, V5, and V6. These waveforms were originally encoded in the base64 format. Each waveform extended across a time span of 10 seconds operating at either 250 or 500 samples per second. These ECG records were recoded using GE ECG machines. To develop a first hyperkalemia model, Applicant elected to first filter the dataset and to exclusively analyze features from Lead I ECG signals (“Lead I”).

Further, Applicants narrowed the Lead I ECG signals to be analyzed to those Lead I ECG signals of Mayo Clinic patients from 1996 and 2022 who had also received at least one (1) serum potassium test within 4 hours before or after an ECG was administered. Such narrowing provided a dataset for which the serum potassium level of the patient could be matched to the Lead I ECG signal of the patient. For this analysis, applicant defined the mean potassium range for a patient considered having severe hyperkalemia as being in the range 7.1-10.6 mEq/L. A time stamp to the nearest minute was available for the blood draws and ECG recordings. For this analysis, the control group was defined to be patients whose serum potassium test showed values of less than 5 mEq/L. The cut-offs of this model were designed to provide clear identification of positive and negative controls.

Thus, a database comprising of Lead I ECG signals from individuals with hyperkalemia and without hyperkalemia was generated.

### Model Development

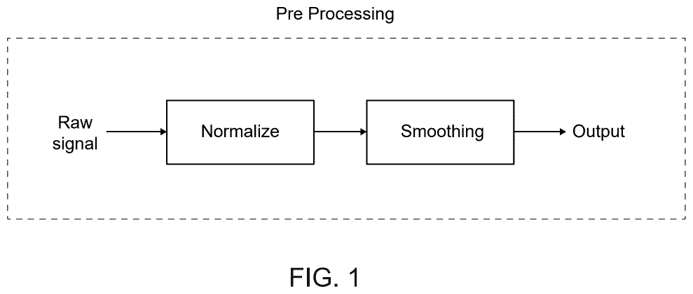

Raw ECG Lead I signal for each of the Lead I ECG signals in the curated database (i.e., ECG signals with a matched serum potassium level taken within 4 hours of the ECG) was cut into smaller, manageable chunks of 5 seconds duration. This segmentation allowed for a more focused examination of specific sections of the signal. Once the ECG signal was segmented, each chunk further underwent a pre-processing technique to improve the quality of the data. The pre-processing technique included normalization of the waveform in the segment by adjusting both the time and amplitude scales. Normalizing the time scale ensured that all segments had a consistent duration, facilitating accurate comparisons and analysis. Similarly, normalizing the amplitude scale ensured that the signal amplitudes were standardized, allowing for meaningful comparisons between different segments. Before feeding the data to training module, the input signal was preprocessed. The processing steps are outlined in FIG. 1, and further described below:

### Signal Pre-Processing:

### Normalize:

The waveform was adjusted across both the time and amplitude scales. Normalizing the time scale ensured that all segments had a consistent duration, and normalizing the amplitude scale ensured that the signal amplitudes are standardized, which facilitates comparisons. The normalization across the time and amplitude scale facilitated accurate comparisons and analysis of different segments of the ECG signal.

### Smoothing:

Following the normalization step, the pre-processed segment underwent a smoothing process to reduce noise and irregularities in the waveform. Here for smoothing the waveform Savitzky-Golay (S-G) filter is used. The S-G filter is a digital filter that effectively reduces noise and unwanted variations in the signal while preserving the underlying prominent features.

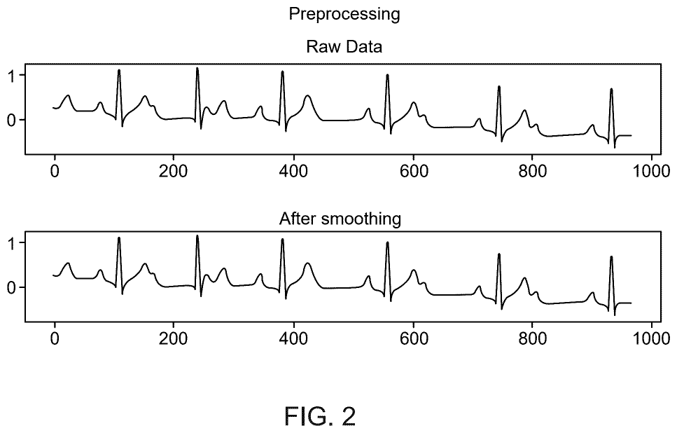

The S-G filter operates by fitting a fixed polynomial order “p” to a local fixed window length (w) of data points and using this polynomial to estimate the smoothed values. The filter takes into account neighboring data points to calculate the smoothed value at each point in the signal. By considering a moving window of data, the S-G filter effectively reduces high-frequency noise and artifacts while preserving the overall shape and characteristics of the signal.
output_wave=savgol_filter(inputwave, window_length=w, polyorder=p)

where both w and p are real values. The S-G filter is particularly advantageous for ECG signals because it can effectively remove noise without significantly distorting the important features such as R-peaks, P-waves, and T-waves. See, e.g. FIG. 2. It provides a balance between noise reduction and preserving the fine details necessary for accurate analysis and interpretation.

The application of the S-G filters as part of the preprocessing stage involves selecting suitable parameters such as the window size (e.g., w=7) and the order of the polynomial (e.g., p=4) used for smoothing. These parameters can be adjusted based on the specific characteristics of the ECG signal and the desired level of smoothing required.

By applying the S-G filter during the pre-processing stage, the ECG waveform is effectively smoothened, resulting in a cleaner and more reliable representation of the underlying prominent feature. FIG. 2 illustrates a high level example of an ECG-waveform before and after smoothing.

### Training Data Preparation:

The training dataset was curated, encompassing exclusively Lead I ECG recordings from adult patients under the care of the Mayo Clinic during the period spanning from 1996 to 2022.

The dataset encompassed only those patients who had undergone a serum potassium test within a 4-hour window before or after the ECG recording. As described above, for model training purposes, compilation of data focused particularly on individuals exhibiting a serum potassium level surpassing 7 mEq/L, thereby indicating the clear presence of hyperkalemia. The patients encompassed in this subset exhibited an average serum potassium level within the severe hyperkalemia range of 7.1 to 10.6 mEq/L. Notably, precise timestamps, accurate to the nearest minute, were associated with both the blood draws and the corresponding ECG recordings.

Concurrently, a control group was chosen to serve as a comparative basis. This control group consisted of patients from the Mayo Clinic's medical practice who displayed serum potassium levels beneath the threshold of 5 mEq/L. The selection criteria ensured that the control group was representative of patients without hyperkalemia. The patients encompassed in this subset exhibited an average serum potassium level within the non-hyperkalemia range of 2.84-4.9 mEq/L.

The model was trained on 1,000 patients and internally tested using the development data set. The Baseline Characteristics of Development set is provided in Table 1 below. A sample of 1000 was collected for training. In this dataset 55% of the patients were identified as male and 44% as female. The data collection process spanned through all the Mayo hospitals including Rochester, Arizona, Florida, and Mayo Clinic hospital system (MCHS). These centers contributed 43.8%, 15%, 20%, and 20% of the dataset, respectively.

TABLE 1
Baseline Characteristics of Development Data set

| Characteristics | All | Hyperkalemia | Non-Hyperkalemia |
|---|---|---|---|
| Training Data Set | 1000 | 500 | 500 |
| Male | 554 | 310 | 244 |
| Female | 446 | 190 | 256 |
| Age, mean (SD) | 55.995 | 55.84 | 56.15 |
| Race/ethnicity | | | |
| White | 831 | 376 | 455 |
| Black | 77 | 62 | 15 |
| Asian | 18 | 18 | — |
| Other | 74 | 44 | 30 |
| Potassium, mean | 5.865 | 7.55 | 4.18 |
| (SD) mEq/L | | | |
| Potassium, range | 2.84-10.6 | 7.1-10.6 | 2.84-4.9 |
| mEq/L | | | |
| Time to potassium | 56.77 | 58.34 | 55.2 |
| test vs ECG, | | | |
| mean in mins | | | |
| Year Range | 1996-2022 | 1996-2022 | 1996-2022 |
| <2010 | 81 | 15 | 66 |
| >2010 | 919 | 485 | 434 |
| Location | | | |
| RST | 438 | 208 | 230 |
| ARZ | 157 | 93 | 64 |
| FLA | 203 | 90 | 113 |
| MCHS | 201 | 109 | 92 |
| OTHER | — | <10 | <10 |
| UNKNOWN | — | <10 | <10 |

### Model Training:

The model was trained on 1,000 patients (see Table 1). The training dataset includes ECG recordings that span a duration of 5 seconds. The training process involved leveraging labelled data from Mayo Clinic to enable accurate detection and analysis of the desired patterns in the ECG signals related with hyperkalemia.

The raw signal was trained for binary classification. The model's compilation was configured to use binary cross-entropy loss, an Adam optimizer, and accuracy as the evaluation metric. The training process incorporated two notable callbacks. The ReduceLROnPlateau callback adjusted the learning rate dynamically based on the plateauing of a monitored metric, which can aid in smoother convergence. The EarlyStopping callback prevents overfitting by monitoring validation metrics; if no significant improvement occurs within a defined patience period, training halts. The model was then trained with specific instructions developed by Applications for 1,000 epochs with these callbacks guiding the process to enhance efficiency and prevent overfitting.

The below code shows the snippet applied for training.
model.compile(loss=‘binary_crossentropy’, optimizer=‘adam’, metrics=[‘accuracy’])
reduce_lr=ReduceLROnPLATEAU( )
early_stopping=EarlyStopping(patience=50, min_delta=0.0001)
model.fit(x,y,epochs=1000,validation_data, callbacks=[reduce_lr,early_stopping]

loss=‘binary_crossentropy’: This parameter specifies the loss function that the model will use during training. In this case, “binary_crossentropy” was employed, but other suitable ones can be considered. This is a suitable choice for binary classification tasks, as it quantifies the disparity between the model's predictions and the true binary labels, guiding the optimization process to minimize this discrepancy.

optimizer=‘adam’: The “optimizer” parameter designates the optimization algorithm responsible for updating the model's weights during training. Here, the Adam optimizer was utilized. Adam adapts the learning rate individually for each parameter, offering a dynamic approach that balances stability and speed in weight updates. This aided in expediting convergence while mitigating the chances of getting stuck in local minima.

metrics=[‘accuracy’]: This parameter specifies the evaluation metric(s) used to assess the model's performance during and after training. In this case, “accuracy” is chosen. Accuracy measures the ratio of correctly predicted instances to the total instances, providing insight into the model's ability to make correct binary classifications.

reduce_lr=ReduceLROnPlateau( ): This callback monitors a specific metric (typically validation loss) during training. If the monitored metric plateaus for a specified number of epochs, the callback reduces the learning rate. This dynamic adjustment aims to aid convergence when progress becomes stagnant, allowing the model to fine-tune its behavior as it approaches a potential optimal solution.

early_stopping=EarlyStopping(patience=50, min_delta=0.0001): The EarlyStopping callback is employed to prevent overfitting. It watches a chosen validation metric and halts training if the metric fails to demonstrate significant improvement over a certain number of epochs, indicated by the “patience” parameter. The “min_delta” parameter determines the minimal change in the monitored metric that qualifies as an improvement, setting a threshold.

model.fit(x, y, validation_data epochs=1000, callbacks=[reduce_lr, early_stopping]): The fit function initiates the training process. It takes in the pre-processed input data x and target labels y. The “epochs” parameter specifies the number of training iterations. The “callbacks” parameter is used to provide the callbacks created earlier, namely reduce_lr and early_stopping. These callbacks were applied during training to adjust the learning rate and possibly halt training early based on predefined conditions, ultimately enhancing the model's convergence and generalization.

After the training process was complete, a final set of weights, representing the weights acquired by the model during this training, were saved.

Although the weights can change from being trained in slightly different models or from changes in the number of datapoints used to train a model; suitable weights were observed in our model with training of 962,434 parameters. It is contemplated that any changes in the number of parameters used to train a model would produce different weights. The weights are thus likely to be applicable to each model created and the number of parameters selected.

### Example 2—Model System Architecture for Hyperkalemia Detection

This example illustrates the architecture of a model system validated for hyperkalemia detection.

A 1-D ResNet model tailored for analyzing sequences, specifically suited for tasks like screening electrocardiogram (ECG) signals was utilized. This architecture leveraged residual blocks, an innovation that facilitated training deeper networks by mitigating the vanishing gradient problem. The model started with an initial convolutional layer, which employed small filters and a stride of 2 to down sample the input. Batch normalization and ReLU activation followed to enhance training stability and non-linearity. The subsequent six residual blocks introduced skip connections, which allow the network to learn residual information effectively. These blocks involve convolutional layers, batch normalization, and activation functions to extract intricate features. The architecture culminated in a global average pooling layer that reduced spatial dimensions, followed by a dense layer with a softmax activation to classify the input into distinct classes. This 1D ResNet design was structured to harness the strengths of residual networks in processing sequential data like ECG signals, making it a robust foundation for various sequence analysis tasks.

In essence, the model transforms input sequences through hierarchical feature extraction, utilizing the residual block's capacity to capture intricate patterns. It amalgamates these features through skip connections, then condenses the information using global average pooling before performing classification through the dense layer. This streamlined yet potent architecture showcases the essence of 1D ResNet's ability to tackle sequential data analysis challenges, particularly well-suited for domains like biomedical signal processing.

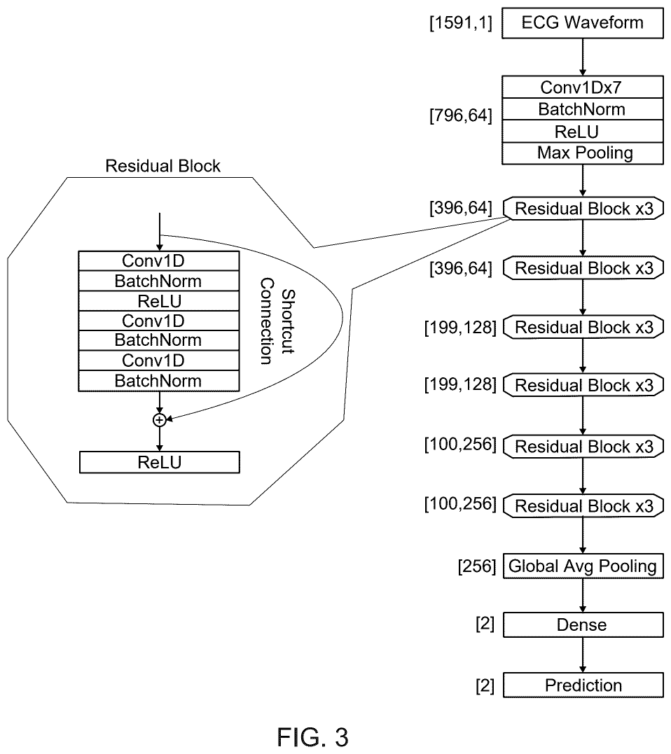

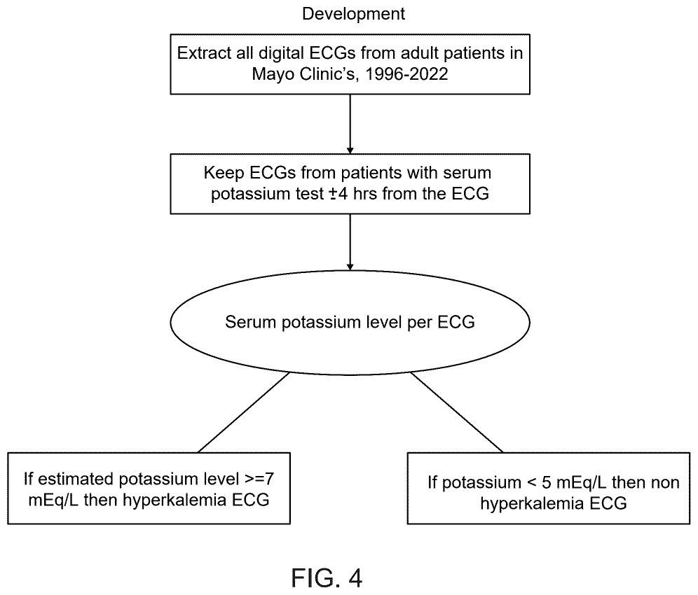

The model architecture has 53 layers and 962,434 parameters. For the hyperkalemia model detection, the network function received a 5-second ECG signal from lead-1 ECG and produced a parameter output between 0 and 1. FIG. 3 depicts the aforementioned ResNet model architecture developed for hyperkalemia detection. Table 2 summarizes the details of the model architecture.

TABLE 2

| | | Number of |
|---|---|---|
| Layer Name | Layer Type | Parameters |
| input_1 | InputLayer | 0 |
| conv1d | Conv1D | 512 |
| batch_normalization | BatchNormalization | 256 |
| activation | Activation | 0 |
| max_pooling1d | MaxPooling1D | 0 |
| conv1d_1 | Conv1D | 12352 |
| batch_normalization_1 | BatchNormalization | 256 |
| activation_1 | Activation | 0 |
| conv1d_2 | Conv1D | 12352 |
| batch_normalization_2 | BatchNormalization | 256 |
| add | Add | 0 |
| activation_2 | Activation | 0 |
| conv1d_3 | Conv1D | 12352 |
| batch_normalization_3 | BatchNormalization | 256 |
| activation_3 | Activation | 0 |
| conv1d_4 | Conv1D | 12352 |
| batch_normalization_4 | BatchNormalization | 256 |
| add_1 | Add | 0 |
| activation_4 | Activation | 0 |
| conv1d_5 | Conv1D | 24704 |
| batch_normalization_5 | BatchNormalization | 512 |
| activation_5 | Activation | 0 |
| conv1d_6 | Conv1D | 49280 |
| conv1d_7 | Conv1D | 8320 |
| batch_normalization_6 | BatchNormalization | 512 |
| batch_normalization_7 | BatchNormalization | 512 |
| add_2 | Add | 0 |
| activation_6 | Activation | 0 |
| conv1d_8 | Conv1D | 49280 |
| batch_normalization_8 | BatchNormalization | 512 |
| activation_7 | Activation | 0 |
| conv1d_9 | Conv1D | 49280 |
| batch_normalization_9 | BatchNormalization | 512 |
| add_3 | Add | 0 |
| activation_8 | Activation | 0 |
| conv1d_10 | Conv1D | 98560 |
| batch_normalization_10 | BatchNormalization | 1024 |
| activation_9 | Activation | 0 |
| conv1d_11 | Conv1D | 196864 |
| conv1d_12 | Conv1D | 33024 |
| batch_normalization_11 | BatchNormalization | 1024 |
| batch_normalization_12 | BatchNormalization | 1024 |
| add_4 | Add | 0 |
| activation_10 | Activation | 0 |
| conv1d_13 | Conv1D | 196864 |
| batch_normalization_13 | BatchNormalization | 1024 |
| activation_11 | Activation | 0 |
| conv1d_14 | Conv1D | 196864 |
| batch_normalization_14 | BatchNormalization | 1024 |
| add_5 | Add | 0 |
| activation_12 | Activation | 0 |
| global_average_pooling1d | GlobalAveragePooling1D | 0 |
| dense | Dense | 514 |

The model architecture described above comprised 53 layers and 962,434 parameters.

### Example 3—Sensitivity and Specificity of Hyperkalemia Detection

Having effectively parsed out ECG signals into Lead I signals that could be classified into signals from patients with hyperkalemia and patients not suffering from hyperkalemia, Applicants set out to further validate a first model created with the process of Examples 1 and 2.

A validation dataset was meticulously curated. Within this validation dataset, five distinct subsets were defined, each bearing a specific association with the presence of hyperkalemia. The first subset consists of 250 patients whose serum potassium levels exceeded the threshold of 7.5 mEq/L, signifying a more pronounced hyperkalemic state. A second subset comprises a count of 500 patients whose potassium levels ranged between 6.5 to 7.5 mEq/L, indicative of a more moderately elevated potassium range. The third subset comprises another 500 patients characterized by potassium levels between 6 to 6.5 mEq/L, further diversifying the scope. Another subset includes 500 patients with potassium levels ranging from 5.5 to 6 mEq/L, providing valuable insights into the ECG patterns associated with a relatively lower yet clinically significant potassium range. This multilayered approach to subgroup definition ensured a comprehensive exploration of the intricate relationship between serum potassium levels and ECG signatures, facilitating a profound understanding of their potential correlation in diverse clinical scenarios that has not been sufficiently explored. For the control non-hyperkalemia group, 20,000 patients characterized by potassium levels lesser than 5 mEq/L were selected.

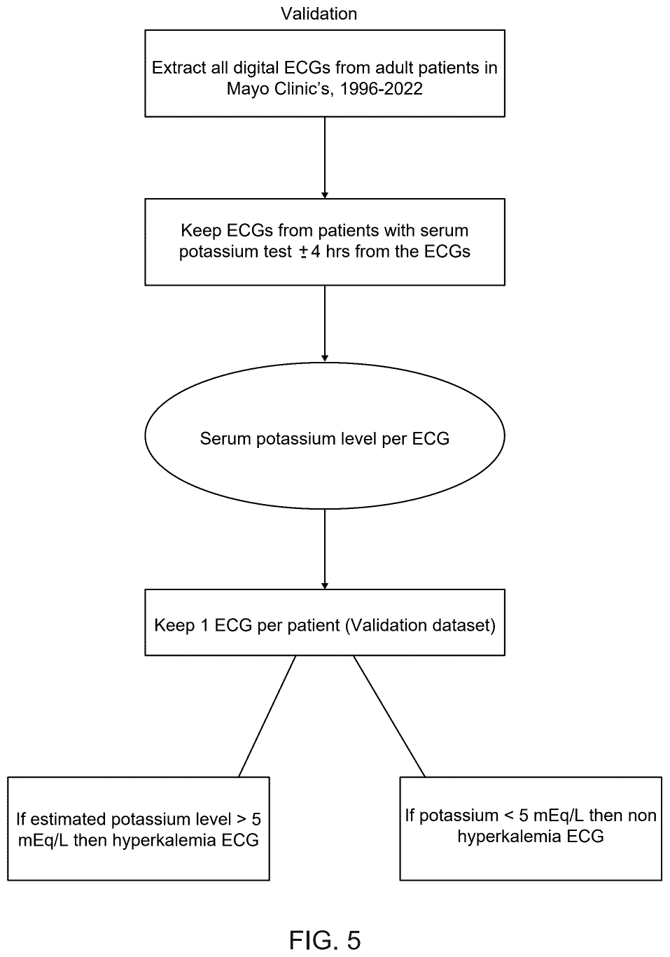

No ECG files part of the training dataset were included in the model validation. FIG. 5 is a flow chart roughly outlining the steps taken for the creation of the validation dataset.

Table 3 (below) provides an overview of the patient demographics within the Validation dataset. A sample of 22,250 was collected for validation. In this dataset 51% of the patients were identified as male and 48% as female. The data collection process spanned through all the Mayo Clinic hospitals including Rochester, Arizona, Florida, and Mayo Clinic hospital system (MCHS). These centers contributed 45.73%, 12.62%, 19.73%, and 21.76% of the dataset, respectively.

TABLE 3

| | | | | | | Non- |
|---|---|---|---|---|---|---|
| Characteristics | A | B | C | D | E | Hyperkalemia |
| Description | >7.5 | >=6.5 & <=7.5 | >=6 & <6.5 | >=5.5 & <6 | >=5 & <5 | <5 |
| Validation Data Set | 250 | 500 | 500 | 500 | 500 | 20000 |
| Male | 150 | 309 | 317 | 295 | 310 | 10020 |
| Female | 100 | 191 | 183 | 205 | 190 | 9979 |
| Non-Binary | — | — | — | — | — | <10 |
| Unknown | — | — | — | — | — | <10 |
| Age, mean (SD) | 56.31 | 59.29 | 62.77 | 63.73 | 63.94 | 57.11 |
| Race/ethnicity | | | | | | |
| White | 209 | 397 | 422 | 415 | 448 | 18331 |
| Black | 27 | 49 | 45 | 36 | 20 | 608 |
| Asian | — | 12 | — | 16 | 10 | 357 |
| Other | 14 | 42 | 33 | 33 | 22 | 704 |
| Potassium, mean (SD) | 7.99 | 6.82 | 6.15 | 5.73 | 5.24 | 4.19 |
| mEq/L | | | | | | |
| Potassium, range mEq/L | [7.6, 9.7] | [6.5, 7.5] | [6.0, 6.4] | [5.53, 5.93] | [5.03.5.41] | [1.8, 4.94] |
| Time to potassium test vs | 57.67 | 82.51 | 88.56 | 85.6 | 72.61 | 55.45 |
| ECG, mean in mins | | | | | | |
| Year Range | [1996, 2022] | [1996, 2022] | [1996, 2022] | [1996, 2022] | [1995, 2022] | [1995, 2022] |
| <2010 | 21 | 41 | 39 | 38 | 60 | 2517 |
| >2010 | 229 | 459 | 461 | 462 | 440 | 17483 |
| Location | | | | | | |
| RST | 113 | 246 | 251 | 264 | 275 | 9027 |
| ARZ | 32 | 77 | 78 | 87 | 66 | 2472 |
| FLA | 33 | 92 | 96 | 73 | 75 | 4021 |
| MCHS | 72 | 84 | 74 | 76 | 84 | 4453 |
| OTHER | <10 | <10 | <10 | <10 | <10 | 27 |
| UNKNOWN | <10 | <10 | <10 | <10 | <10 | — |

### Model Performance:

A sample size of 22,250 ECGs was used for validation. The model was also evaluated at 2 operating points selected from the development data set, one selected for equal sensitivity and specificity and the other for high (90%) sensitivity. These thresholds were applied to the validation data sets to characterize the sensitivity and specificity of the algorithm. Exact 95% confidence intervals were used for all measures of diagnostic performance except for AUC. The confidence interval for AUC was determined based on Sun and Su optimization of the Delong method.

### Results

Performance of the model was considered under a range of defined parameters. In one evaluation, results with a confidence level above 0.5 were considered indicative of hyperkalemia. Anything else not considered hyperkalemia. The algorithm performed well in identifying hyperkalemia in the validation data sets. The validation was first conducted on 5 subset which was grouped according to the serum potassium. In the subset where the serum potassium level exceeded 7.5, the algorithm demonstrated an Area Under the Curve (AUC) of 0.917, with a 95% Confidence Interval (CI) ranging from 0.896 to 0.935. Similarly, when serum potassium range of 6.5 to 7.5, the AUC was measured at 0.870 (95% CI, 0.861-0.891). For range of 6 to 6.5, the algorithm had an AUC of 0.822 (95% CI, 0.805-0.839). Further, in the serum potassium range between 5.5 and 6, the AUC remained substantial at 0.784 (95% CI, 0.761-0.805). Lastly, within the range of 5 to 5.5 serum potassium levels, the algorithm exhibited an AUC of 0.717 (95% CI, 0.693-0.741). These results collectively underscore the algorithm's ability to effectively discern hyperkalemia across varying levels of serum potassium concentrations.

Table 4 summarizes validation data set performance for Hyperkalemia at different K values under the aforementioned scenario.

TABLE 4

| | SE | SP | PPV | NPV | AUC |
|---|---|---|---|---|---|
| | K > 7.5, K < 5 | | | | |
| Result | 89.6 | 83.2 | 6.25 | 99.84 | 91.79 |
| | (85.8-93.38) | (82.69-83.72) | (5.46-7.04) | (99.78-99.9) | (89.68-93.56) |
| SE = SP | 86 | 86.05 | 7.15 | 99.79 | 86.03 |
| | (81.7-90.3) | (85.57-86.54) | (6.24-8.08) | (99.73-99.86) | (83.78-88.17) |
| 90% SE | 90 | 81.83 | 5.83 | 99.84 | 85.92 |
| | (86.28-93.72) | (81.3-82.37) | (5.09-6.57) | (99.79-99.91) | (83.83-87.62) |
| | K >= 6.5 + K <= 7.5 | | | | |
| Result | 74.6 | 83.2 | 9.99 | 99.24 | 87.69 |
| | ( 70.78-78.42 ) | (82.69-83.72) | ( 9.03-10.96 ) | ( 99.11-99.37 ) | ( 86.15-89.17 ) |
| SE = SP | 79.4 | 79.34 | 8.77 | 99.36 | 79.37 |
| | ( 75.86-82.94 ) | ( 78.78-79.91 ) | ( 7.94-9.59 ) | ( 99.23-99.48 ) | ( 77.62-81.2) |
| 90% SE | 90.0 | 67.07 | 6.4 | 99.63 | 78.52 |
| | ( 87.37-92.63 ) | ( 66.42-67.72 ) | ( 5.82-6.97 ) | ( 99.53-99.73 ) | ( 77.15-79.85 ) |
| | K >= 6 + K < 6.5, K < 5 | | | | |
| Result | 64.2 | 83.2 | 8.72 | 98.94 | 82.26 |
| | ( 60.0-68.4) | ( 82.69-83.72 ) | ( 7.81-9.63 ) | ( 98.78-99.09 ) | ( 80.58-83.89 ) |
| SE = SP | 75.2 | 75.1 | 7.02 | 99.18 | 75.15 |
| | ( 71.41-78.99 ) | ( 74.5-75.69 ) | ( 6.33-7.7) | ( 99.04-99.32 ) | ( 73.16-77.05 ) |
| 90% SE | 90.0 | 55.84 | 4.85 | 99.55 | 72.92 |
| | ( 87.37-92.63 ) | ( 55.16-56.53 ) | ( 4.41-5.29 ) | ( 99.43-99.68 ) | ( 71.52-74.27 ) |
| | K >= 5.5 + K < 6, K < 5 | | | | |
| Result | 60.8 | 83.2 | 8.3 | 98.84 | 78.41 |
| | ( 56.52-65.08 ) | ( 82.69-83.72 ) | ( 7.41-9.19 ) | ( 98.67-99.0) | ( 76.14-80.49 ) |
| SE = SP | 71.40 | 71.59 | 5.91 | 99.00 | 71.5 |
| | ( 67.44-75.36 ) | ( 70.96-72.22 ) | (5.32-6.51 ) | ( 98.85-99.17) | ( 69.42-73.58 ) |
| 90% SE | 90.0 | 38.68 | 3.54 | 99.36 | 64.34 |
| | ( 87.37-92.63 ) | ( 38.0-39.35 ) | ( 3.22-3.86 ) | ( 99.18-99.54 ) | ( 62.9-65.68 ) |
| | K >= 5 + K < 5.5, K < 5 | | | | |
| Result | 47.8 | 83.2 | 6.64 | 98.46 | 71.77 |
| | ( 43.42-52.18 ) | ( 82.69-83.72 ) | ( 5.83-7.46 ) | ( 98.27-98.64 ) | ( 69.36-74.09 ) |
| SE = SP | 66.8 | 66.52 | 4.75 | 98.77 | 66.66 |
| | ( 62.67-70.93 ) | ( 65.86-67.17 ) | ( 4.25-5.25 ) | ( 98.58-98.95 ) | ( 64.66-68.76 ) |
| 90% SE | 90.0 | 25.46 | 2.93 | 99.03 | 57.74 |
| | ( 87.37-92.63 ) | ( 24.85-26.06 ) | ( 2.66-3.2) | ( 98.76-99.3 ) | ( 56.36-59.09 ) |

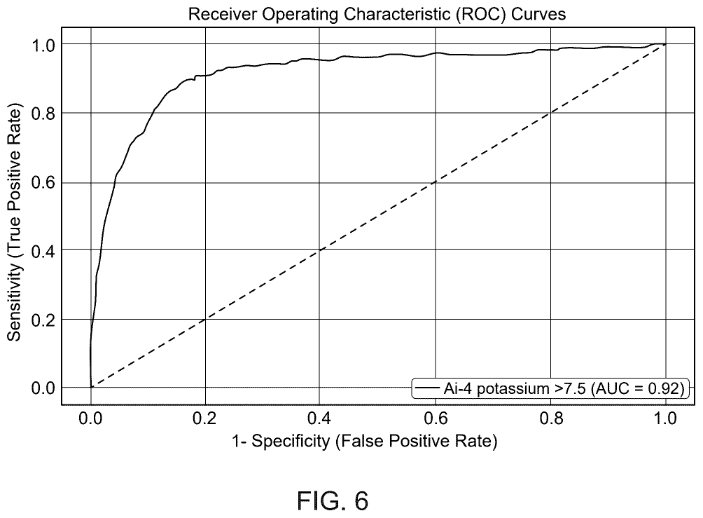

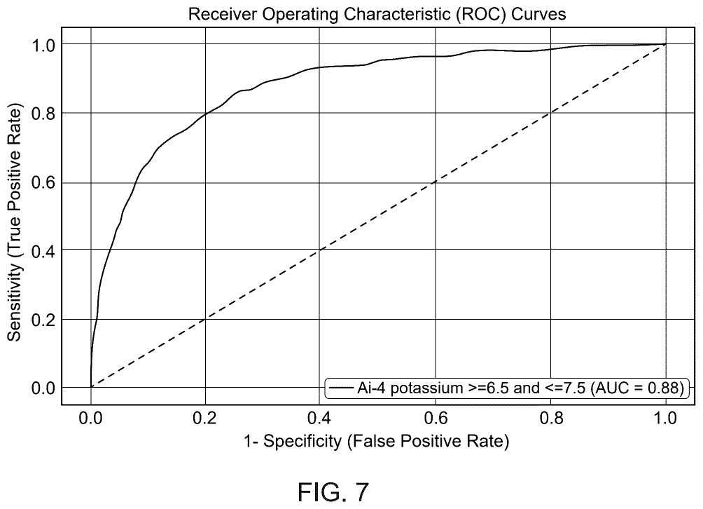

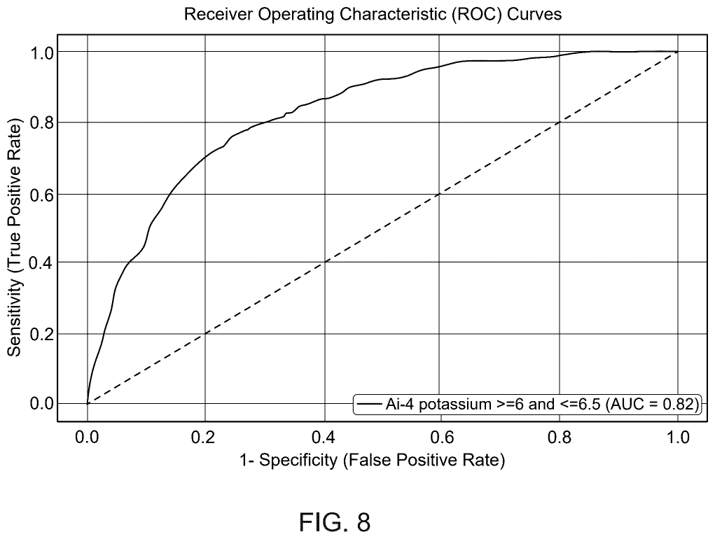

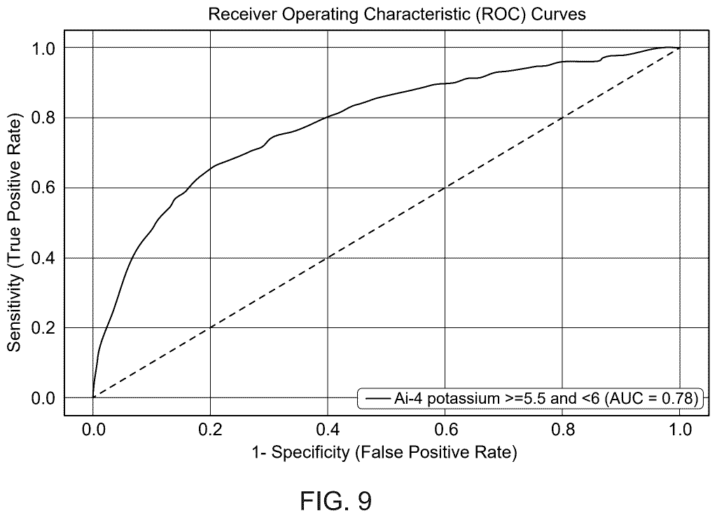

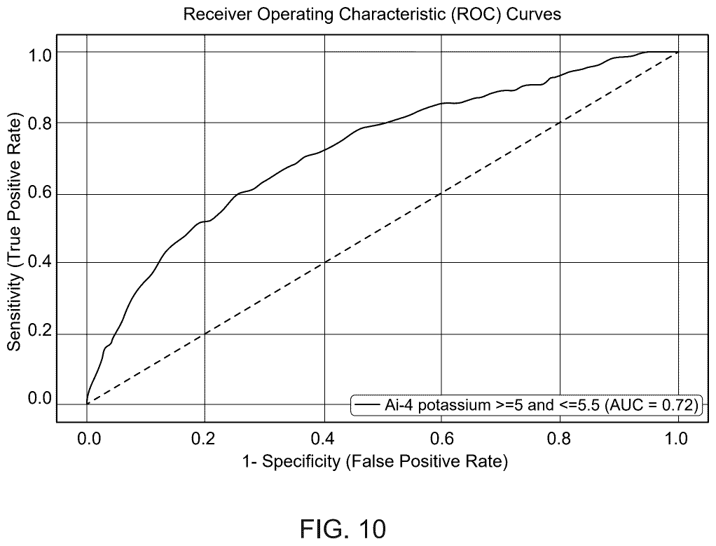

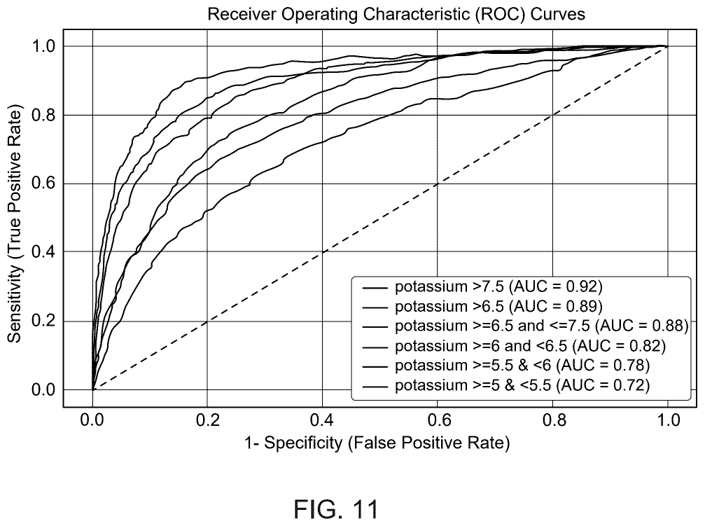

AUC indicates area under the receiver operating characteristic curve for all the test cases. A graphical representation of the data on Table 4 can be found on FIGS. 6-11.

Performance of the model was also considered in a different study; defining the specificity and sensitivity at the same point, or at fixed points for one parameter but not the other.

Utilizing the operating point characterized by equal sensitivity and specificity, the performance of the DNN in predicting hyperkalemia through ECG using lead-1 was as follows for each respective subset. When the serum potassium level is greater than 7.5, the sensitivity and specificity were 86% (95% CI, 81.7%-90.3%) and 86.05% (95% CI, 85.57%-86.54%), respectively. Similarly, for cases within the serum potassium range of 6.5 to 7.5, the sensitivity and specificity exhibited values of 79.4% (95% CI, 75.86%-82.94%) and 79.34% (95% CI, 78.78%-79.91%). As the serum potassium range narrowed to 6 to 6.5, the algorithm maintained a sensitivity of 75.2% (95% CI, 71.41%-78.99%) and a specificity of 75.1% (95% CI, 74.5%-75.69%). Progressing further, within the serum potassium range of 5.5 to 6, the algorithm demonstrated a sensitivity of 71.40% (95% CI, 67.44%-75.36%) alongside a specificity of 71.49% (95% CI, 70.96%-72.22%). Lastly, for serum potassium levels in the range of 5 to 5.5, the algorithm exhibited a sensitivity of 66.8% (95% CI, 62.67%-70.93%) and a specificity of 66.62% (95% CI, 65.86%-67.17%).

Utilizing the operating point characterized by high sensitivity, the performance of the model is indicative of an output suitable for a screening tool. When the serum potassium level exceeds 7.5, the algorithm demonstrated a sensitivity of 90% (95% CI, 86.28-93.72) alongside a specificity of 81.83% (95% CI, 81.3-82.37). Similarly, when serum potassium range of 6.5 to 7.5, the sensitivity and specificity attained values of 90% (95% CI, 87.37-92.63%) and 67.07% (95% CI, 66.42-67.72%). As the serum potassium range narrows to 6 to 6.5, the algorithm consistently upheld a sensitivity of 90% (95% CI, 87.37-92.63%) accompanied by a specificity of 55.84% (95% CI, 55.16-56.53). Progressing further, within the serum potassium range of 5.5 to 6, the algorithm showcased a sensitivity of 90% (95% CI, 87.37-92.63%) along with a specificity of 38.68% (95% CI, 38.0-39.35). Lastly, for serum potassium levels spanning from 5 to 5.5, the algorithm exhibited a sensitivity of 90% (95% CI, 87.37-92.63%) paired with a specificity of 25.46% (95% CI, 24.85-26.06%).

The number of false-positive, false-negative, true-positive, and true-negative results for each model, as well as accuracy, is presented in Table5.

Table 5 Confusion Matrix for Classification of Hyperkalemia for different K values

TABLE 5

| | TP | TN | FP | FN | Accuracy |
|---|---|---|---|---|---|
| | K > 7.5, K < 5 | | | | |
| Result | 224 | 16641 | 3359 | 26 | 83.28 |
| SE = SP | 215 | 17211 | 2789 | 35 | 86.05 |
| 90% SE | 225 | 16367 | 3633 | 25 | 81.93 |
| | K >= 6.5 + K <= 7.5, K < 5 | | | | |
| Result | 373 | 16641 | 3359 | 127 | 82.99 |
| SE = SP | 397 | 15869 | 4131 | 103 | 79.34 |
| 90% SE | 450 | 13414 | 6586 | 50 | 67.62 |
| | K >= 6 + K < 6.5, K < 5 | | | | |
| Result | 321 | 16641 | 3359 | 179 | 82.74 |
| SE = SP | 376 | 15019 | 4981 | 124 | 75.09 |
| 90% SE | 450 | 11169 | 8831 | 50 | 56.67 |
| | K >= 5.5 + K < 6, K < 5 | | | | |
| Result | 304 | 16641 | 3359 | 196 | 82.65 |
| SE = SP | 357 | 14318 | 5682 | 143 | 71.58 |
| 90% SE | 450 | 7735 | 12262 | 50 | 39.92 |
| | K >= 5 + K < 5.5, K < 5 | | | | |
| Result | 239 | 16641 | 3359 | 261 | 82.34 |
| SE = SP | 334 | 13303 | 6697 | 166 | 66.52 |
| 90% SE | 450 | 5091 | 14909 | 50 | 27.02 |

As the concentration of potassium in the bloodstream decreases, a noticeable trend emerges in which the sensitivity, specificity, and the area under the receiver operating characteristic curve (AUC) all experience a gradual decline. This phenomenon is particularly pronounced in the context of medical assessments reliant on potassium-sensitive indicators. Specifically focusing on sensitivity, a crucial diagnostic metric, its performance diminishes concomitantly with decreasing potassium levels. In essence, the ability of a test or diagnostic model to correctly identify true positive cases dwindles as potassium concentrations decline, potentially leading to a higher rate of false negatives and a reduced ability to detect instances of interest. AUC indicates area under the receiver operating characteristic curve for all the test cases.

### Example 4—Model Performance of Patients with Acute Kidney Disease

Applicants set out to further validate model performance on patients with acute kidney disease.

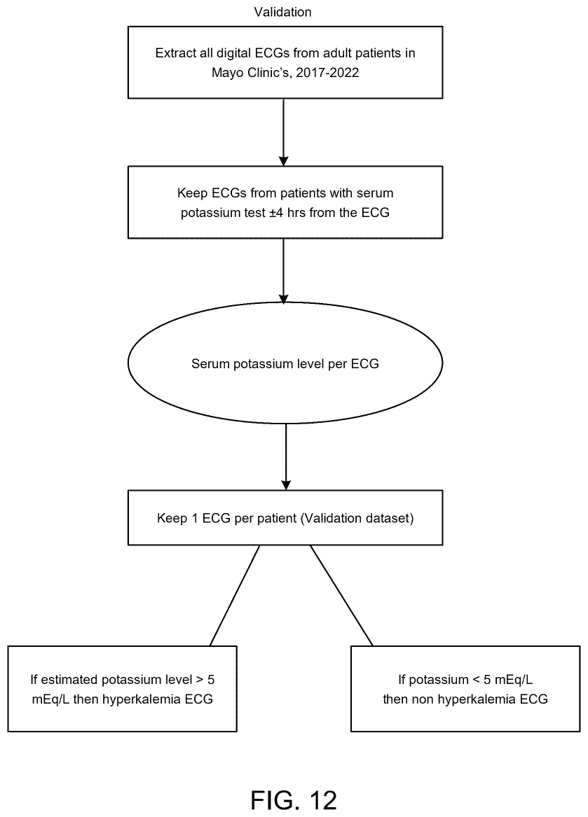

A validation dataset was meticulously curated, with the lead I electrocardiogram (ECG) recordings from adult patients under the care of the Mayo Clinic during the period spanning from 2017 to 2022. See FIG. 12. The validation dataset has patients who had undergone a serum potassium test within a 4-hour window before or after the ECG recording.

Within this validation dataset, five distinct subsets were defined, each bearing a specific association with the presence of hyperkalemia. The first subset consists of 33 patients whose serum potassium levels exceeded the threshold of 7.5 mEq/L, signifying a pronounced hyperkalemic state. A second subgroup comprises a count of 210 patients whose potassium levels ranged between 6.5 to 7.5 mEq/L, indicative of a moderately elevated potassium range. The third subgroup involves another 388 patients characterized by potassium levels between 6 to 6.5 mEq/L, further diversifying the investigation's scope. The fourth subset includes 1091 patients with potassium levels ranging from 5.5 to 6 mEq/L, providing valuable insights into the ECG patterns associated with a relatively lower yet clinically significant potassium range. The Final subset includes 4972 patients with potassium levels ranging from 5 to 5.5 mEq/L, providing valuable insights into the ECG patterns associated with a relatively lower yet clinically significant potassium range. This multilayered approach to subgroup definition ensures a comprehensive exploration of the intricate relationship between serum potassium levels and ECG signatures, facilitating a profound understanding of their potential correlation in diverse clinical scenarios. As for non-hyperkalemia, 20000 patients characterized by potassium levels less than 5 mEq/L were selected. FIG. 12 is a flow chart depicting steps of the method used in the validation of the dataset used in this example.

Table 6 provides an overview of the patient demographics within the Validation dataset. A sample of 26694 was collected for validation.

TABLE 6

| | | | | | | Non- |
|---|---|---|---|---|---|---|
| Characteristics | A | B | C | D | E | Hyperkalemia |
| Potassium | >7.5 | >=6.5 | >=6 | >=5.5 | >=5 | <5 |
| Level | | & <=7.5 | & <6.5 | & <6 | & <5.5 | |
| Validation | 33 | 210 | 388 | 1091 | 4972 | 20000 |
| Data Set | | | | | | |
| Year Range | [2017-2022] | [2017-2022] | [2017-2022] | [2017-2022] | [2017-2022] | [2017-2022] |

### Model Performance:

A sample size of 26694 ECGs was used for validation. The model was evaluated at 2 operating points selected from the development data set, one selected for equal sensitivity and specificity and the other for high (90%) sensitivity. These thresholds were applied to the validation data sets to characterize the sensitivity and specificity of the algorithm. Exact 95% confidence intervals were used for all measures of diagnostic performance except for AUC. The confidence interval for AUC was determined based on Bootstrap resampling.

### Results

The algorithm performed well in identifying hyperkalemia in the validation data sets. The validation was conducted on 5 subsets which were grouped according to the serum potassium. In the subset where the serum potassium level exceeded 7.5, the algorithm demonstrated an Area Under the Curve (AUC) of 86.71, with a 95% Confidence Interval (CI) ranging from 86.65 to 86.71. Similarly, when serum potassium range of 6.5 to 7.5, the AUC was measured at 86.6 (95% CI, 86.58-86.60). For range of 6 to 6.5, the algorithm had an AUC of 77.38 (95% CI 77.36-77.38). Further, in the serum potassium range between 5.5 and 6, the AUC remained substantial at 69.77 (95% CI, 69.75-69.77). Lastly, within the range of 5 to 5.5 serum potassium levels, the algorithm exhibited an AUC of 58.19 (95% CI, 58.18-58.19). These results collectively underscore the algorithm's ability to effectively discern hyperkalemia across varying levels of serum potassium concentrations.

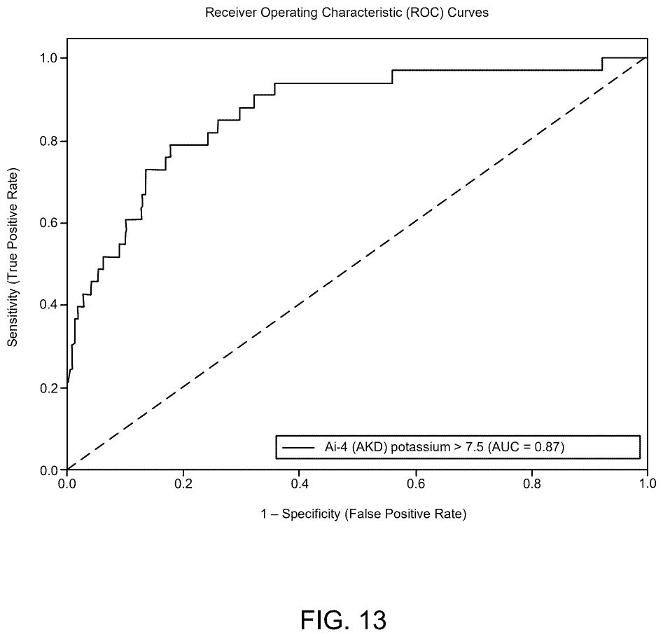

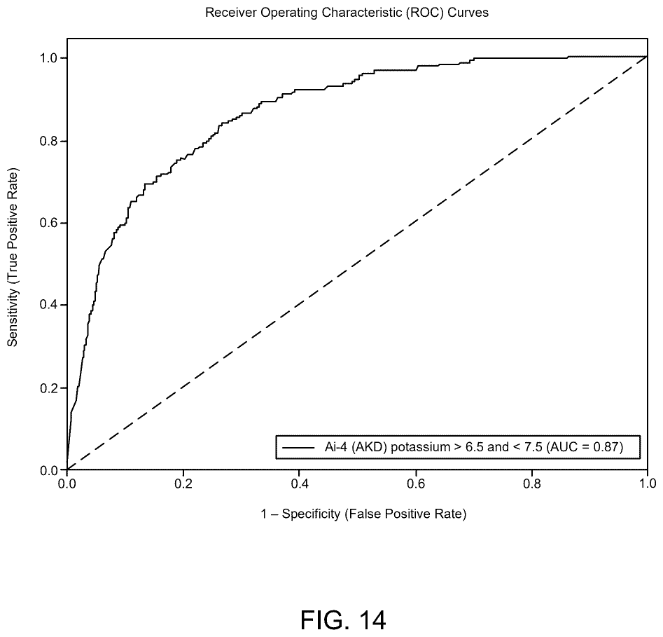

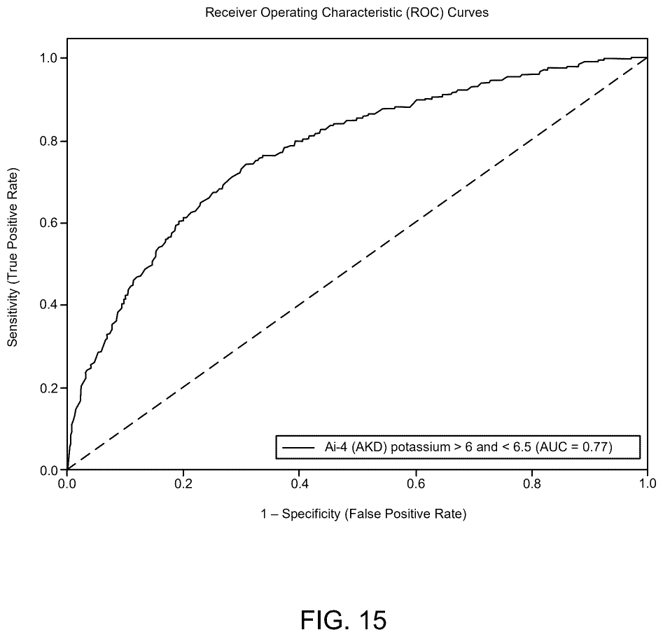

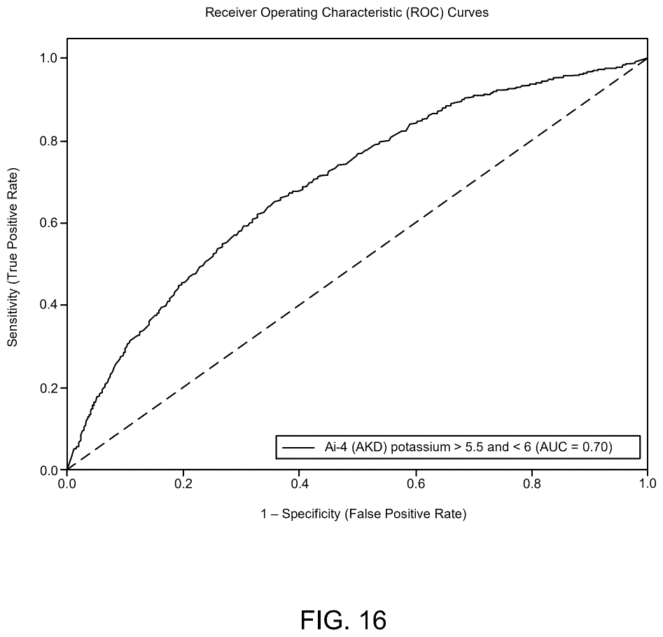

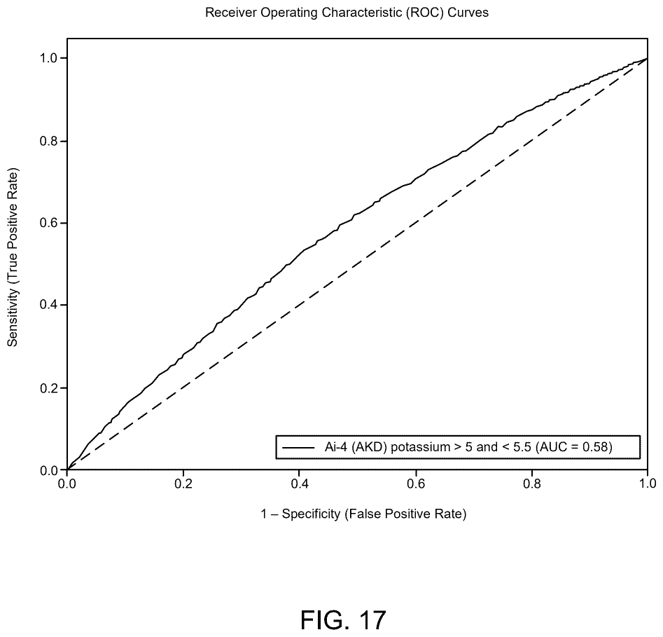

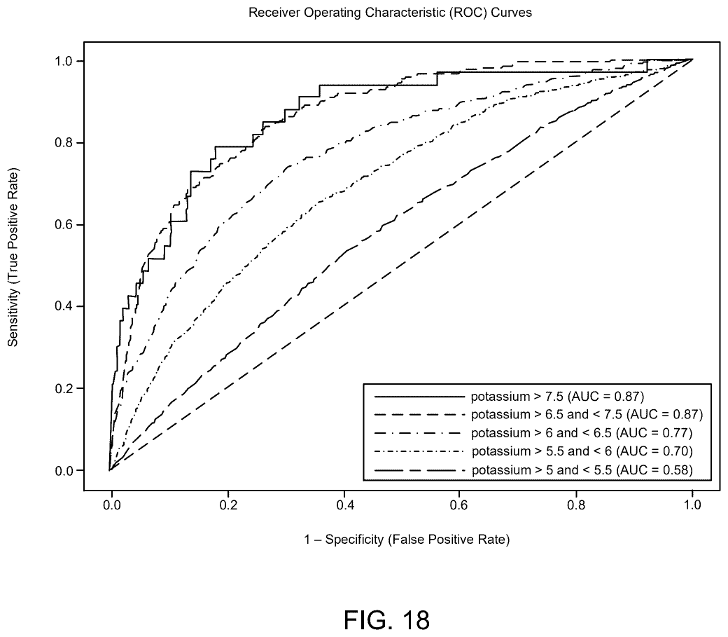

Table 7 summarizes validation data set performance for Hyperkalemia at different K values. A graphical representation of the data on Table 7 can be found on FIGS. 13-18.

TABLE 7

| | | SE | SP | PPV | NPV | AUC |
|---|---|---|---|---|---|---|
| K > 7.5, K < 5 | Result | 78.79 | 76.42 | 0.55 | 99.95 | 86.71 |
| | | (64.84-92.74) | (75.83-77.01) | (0.34-0.76) | (99.92-99.99) | (86.65-86.71) |
| | SE = SP | 78.79 | 78.79 | 0.61 | 99.96 | 74.31 |
| | | (64.84-92.74) | (78.22-79.36) | (0.38-0.84) | (99.92-99.99) | (74.29-74.31) |
| | SE = 90 | 90.91 | 67.78 | 0.46 | 99.98 | 65.13 |
| | | (81.10-100) | (67.13-68.42) | (0.3-0.63) | (99.95-100.0) | (65.11-65.13) |
| K >= 6.5 + K <= 7.5, | Result | 79.05 | 76.42 | 3.40 | 99.71 | 86.6 |
| K < 5 | | (73.54-84.55) | (75.83-77.01) | (2.89-3.91) | (99.63-99.8) | (86.58-86.60) |
| | SE = SP | 77.62 | 77.62 | 3.51 | 99.7 | 73.82 |
| | | (71.98-83.26) | (77.04-78.2) | (2.98-4.04) | (99.61-99.78) | (73.79-73.82) |
| | SE = 90 | 90.0 | 64.02 | 2.56 | 99.84 | 62.28 |
| | | (85.94-94.06) | (63.35-64.68) | (2.19-2.92) | (99.77-99.91) | (62.27-62.29) |
| K >= 6 + K < 6.5, | Result | 65.21 | 76.42 | 5.09 | 99.12 | 77.38 |
| K < 5 | | (60.47-69.95) | (75.83-77.01) | (4.48-5.7) | (98.98-99.27) | (77.36-77.38) |
| | SE = SP | 71.39 | 71.3 | 4.6 | 99.23 | 63.83 |
| | | (66.89-75.89) | (70.67-71.92) | (4.07-5.13) | (99.08-99.37) | (63.81-63.83) |
| | SE = 90 | 90.21 | 37.77 | 2.74 | 99.5 | 36.27 |
| | | (87.25-93.16) | (37.1-38.45) | (2.45-3.02) | (99.34-99.66) | (36.25-36.27) |
| K >= 5.5 + K < 6, | Result | 50.6 | 76.42 | 10.48 | 96.59 | 69.77 |
| K < 5 | | (47.63-53.56) | (75.83-77.01) | (9.65-11.31) | (96.31-96.88) | (69.75-69.77) |
| | SE = SP | 64.8 | 64.8 | 9.13 | 97.12 | 55.97 |
| | | (61.97-67.64) | (64.14-65.47) | (8.49-9.77) | (96.84-97.41) | (55.95-55.97) |
| | SE = 90 | 90.10 | 32.37 | 6.78 | 98.36 | 30.77 |
| | | (88.33-91.87) | (31.72-33.02) | (6.37-7.18) | (98.05-98.67) | (30.75-30.78) |
| K >= 5 + K < 5.5, | Result | 32.18 | 76.42 | 25.33 | 81.93 | 58.19 |
| K < 5 | | (30.88-33.48) | (75.83-77.01) | (24.26-26.41) | (81.37-82.48) | (58.18-58.19) |
| | SE = SP | 56.21 | 56.22 | 24.19 | 83.78 | 45.06 |
| | | (54.84-57.59) | (55.53-56.89) | (23.41-24.98) | (83.15-84.39) | (45.05-45.06) |
| | SE = 90 | 90.0 | 16.61 | 21.16 | 86.99 | 15.82 |
| | | (89.17-90.84) | (16.09-17.13) | (20.61-21.71) | (85.92-88.05) | (15.80-15.83) |

### Example 5—Model Performance of Patients with Chronic Kidney Disease

Applicants set out to further validate model performance on patients with acute kidney disease.

A validation dataset was meticulously curated, with the lead I electrocardiogram (ECG) recordings from adult patients under the care of the Mayo Clinic during the period spanning from 1996 to 2022. The validation dataset has patients who had undergone a serum potassium test within a 4-hour window before or after the ECG recording.

Within this validation dataset, five distinct subsets were defined, each bearing a specific association with the presence of hyperkalemia. The first subset consists of 231 patients whose serum potassium levels exceeded the threshold of 7.5 mEq/L, signifying a pronounced hyperkalemic state. A second subgroup comprises a count of 1517 patients whose potassium levels ranged between 6.5 to 7.5 mEq/L, indicative of a moderately elevated potassium range. The third subgroup involves another 2440 patients characterized by potassium levels between 6 to 6.5 mEq/L, further diversifying the investigation's scope. The fourth subset includes 3942 patients with potassium levels ranging from 5.5 to 6 mEq/L, providing valuable insights into the ECG patterns associated with a relatively lower yet clinically significant potassium range. The fifth subset includes 6120 patients with potassium levels ranging from 5 to 5.5 mEq/L. This multilayered approach to subgroup definition ensures a comprehensive exploration of the intricate relationship between serum potassium levels and ECG signatures, facilitating a profound understanding of their potential correlation in diverse clinical scenarios. were selected. Exemplary flow charts depicting illustrative steps for the validation of the dataset have been provided in prior examples.

Table 8 provides an overview of the patient demographics within the Validation dataset. A sample of 34,250 was collected for validation.

TABLE 8

| | | | | | | Non- |
|---|---|---|---|---|---|---|
| Characteristics | A | B | C | D | E | Hyperkalemia |
| Description | >7.5 | >=6.5 | >=6 | >=5.5 | >=5 | <5 |
| | | & <=7.5 | & <6.5 | & <6 | & <5 | |
| Validation | 231 | 1517 | 2440 | 3942 | 6120 | 20000 |
| Data Set | | | | | | |
| Year Range | [1996-2022] | [1996-2022] | [1996-2022] | [1996-2022] | [1996-2022] | [1996-2022] |

### Model Performance:

A sample size of 34,250 ECGs was used for validation. The model was evaluated at 2 operating points selected from the development data set, one selected for equal sensitivity and specificity and the other for high (90%) sensitivity. These thresholds were applied to the validation data sets to characterize the sensitivity and specificity of the algorithm. Exact 95% confidence intervals were used for all measures of diagnostic performance except for AUC. The confidence interval for AUC was determined based on Bootstrap resampling.

### Results

The algorithm performed well in identifying hyperkalemia in the validation data sets. The validation was conducted on 5 subset which was grouped according to the serum potassium. In the subset where the serum potassium level exceeded 7.5, the algorithm demonstrated an Area Under the Curve (AUC) of 88.18, with a 95% Confidence Interval (CI) ranging from 88.14 to 88.18. Similarly, when serum potassium range of 6.5 to 7.5, the AUC was measured at 79.29 (95% CI, 79.27-79.29). For range of 6 to 6.5, the algorithm had an AUC of 74.33 (95% CI, 74.32-74.33). Further, in the serum potassium range between 5.5 and 6, the AUC remained substantial at 69.77 (95% CI, 69.76-69-78). Lastly, within the range of 5 to 5.5 serum potassium levels, the algorithm exhibited an AUC of 64.84 (95% CI, 64.83-64.85). These results collectively underscore the algorithm's ability to effectively discern hyperkalemia across varying levels of serum potassium concentrations.

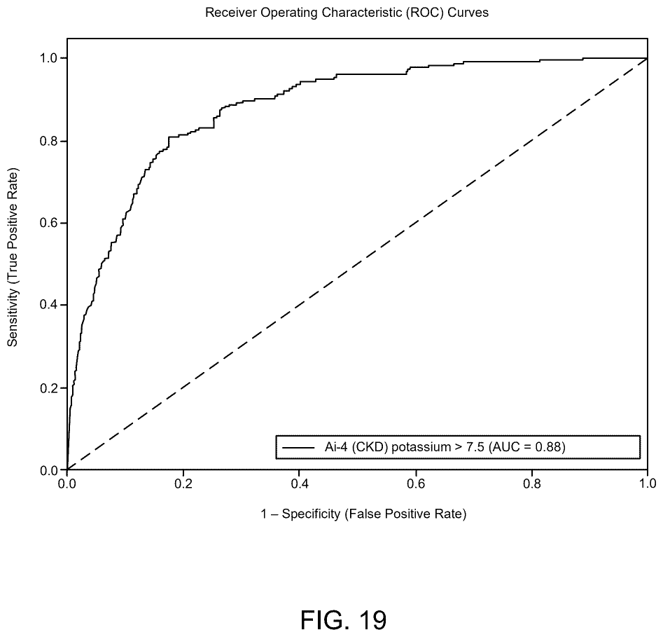

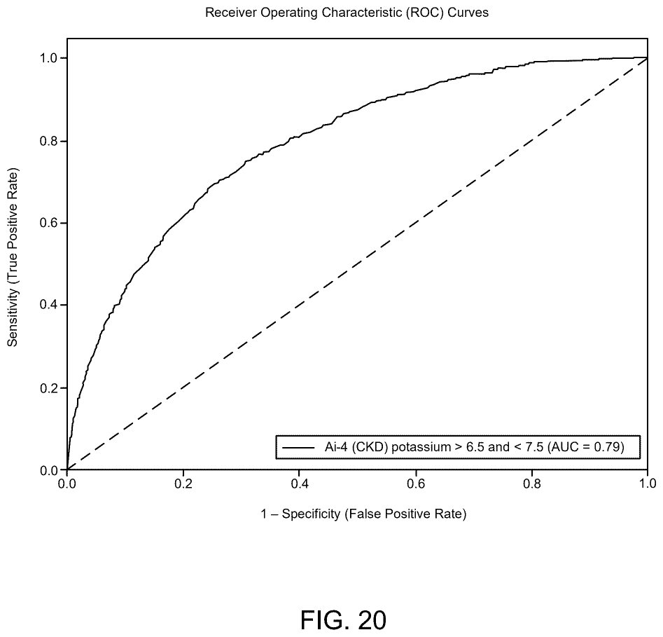

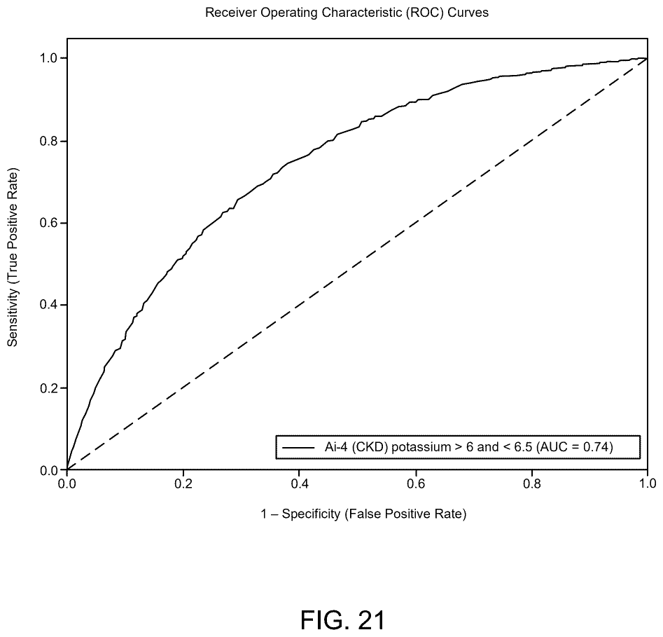

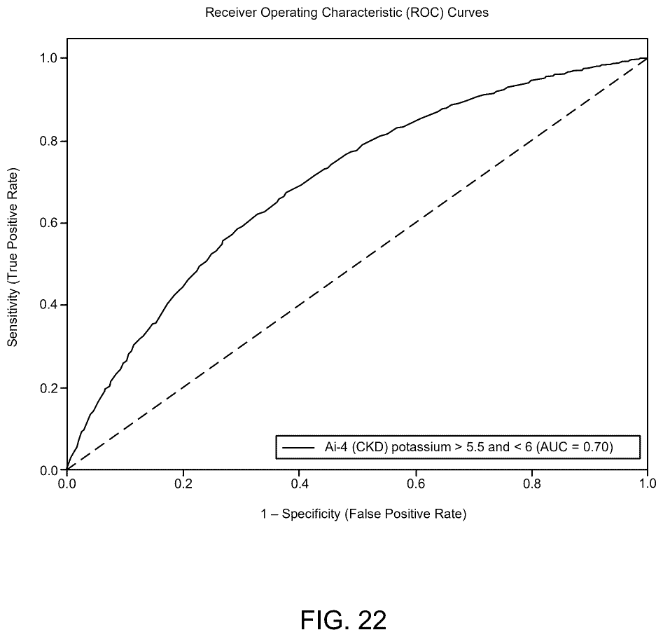

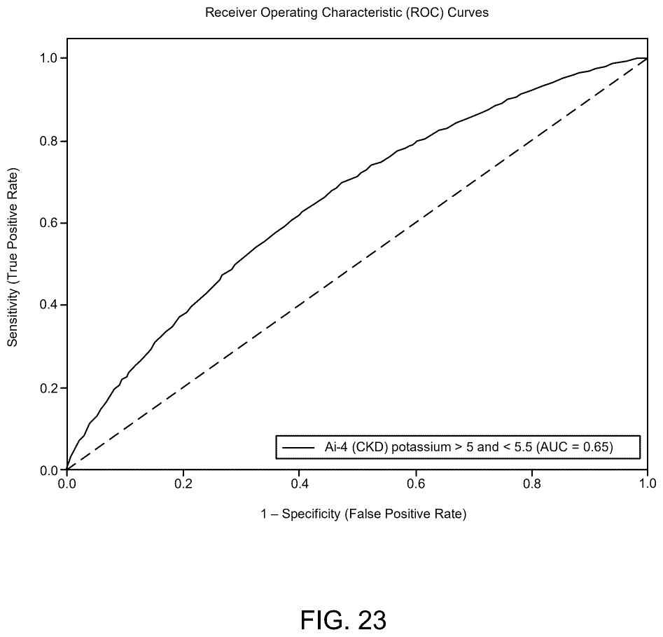

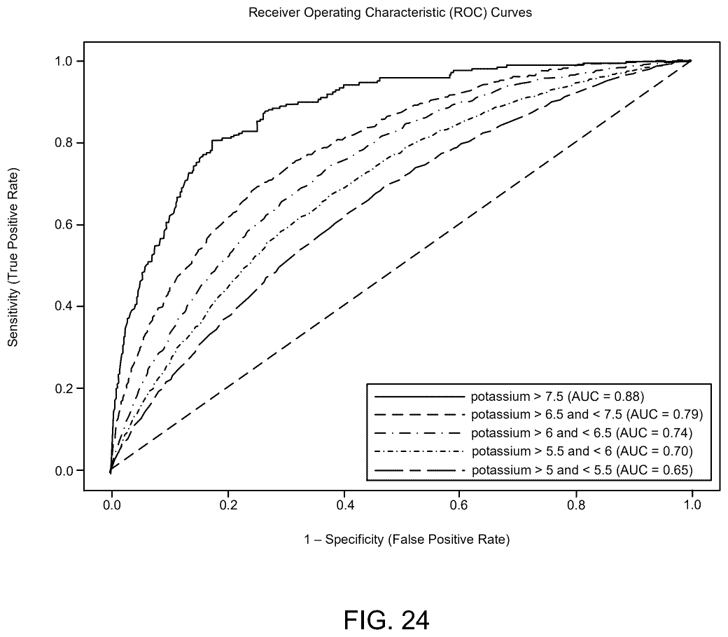

Table 9 provides validation data set performance for Hyperkalemia at different K values. A graphical representation of the data on Table 9 can be found on FIGS. 19-24.

TABLE 9

| | SE | SP | PPV | NPV | AUC |
|---|---|---|---|---|---|
| | K > 7.5, K < 5 | | | | |
| Result | 89.61 | 68.12 | 3.14 | 99.82 | 88.18 |
| | ( 85.68-93.55 ) | ( 67.47-68.76 ) | ( 2.72-3.57) | ( 99.75-99.89 ) | (88.14-88.18) |
| SE = SP | 80.95 | 80.95 | 4.68 | 99.73 | 77.24 |
| | ( 75.89-86.02 ) | ( 80.41-81.49 ) | ( 4.02-5.33 ) | ( 99.65-99.81 ) | (77.22-77.24) |
| 90% SE | 90.04 | 67.86 | 3.13 | 99.83 | 66.03 |
| | ( 86.18-93.89 ) | ( 67.21-68.51 ) | ( 2.72-3.55 ) | ( 99.76-99.9 ) | (66.01, 66.03) |
| | K >= 6.5 + K <= 7.5 | | | | |
| Result | 75.68 | 68.12 | 15.26 | 97.36 | 79.29 |
| | ( 73.52-77.83 ) | ( 67.47-68.76 ) | ( 14.44-16.07 ) | ( 97.1-97.63 ) | (79.27, 79.29) |
| SE = SP | 71.72 | 71.64 | 16.09 | 97.09 | 65.57 |
| | ( 69.45-73.99 ) | ( 71.02-72.26 ) | ( 15.22-16.97 ) | ( 96.82-97.36 ) | (65.55, 65.57) |
| 90% SE | 90.05 | 45.80 | 11.19 | 98.38 | 44.42 |
| | ( 88.54-91.55 ) | ( 45.11-46.5) | ( 10.63-11.75 ) | ( 98.12-98.63 ) | (44.40,4 4.43) |
| | K >= 6 + K < 6.5, K < 5 | | | | |
| Result | 67.62 | 68.12 | 20.56 | 94.52 | 74.33 |
| | ( 65.77-69.48 ) | ( 67.47-68.76 ) | ( 19.67-21.44 ) | ( 94.15-94.89 ) | (74.32, 74.33) |
| SE = SP | 67.95 | 67.92 | 20.54 | 94.56 | 60.78 |
| | ( 66.10-69.8) | ( 67.27-68.57 ) | ( 19.65-21.42) | ( 94.19-94.93 ) | (60.76, 60.78) |
| 90% SE | 90.0 | 38.42 | 15.13 | 96.92 | 37.00 |
| | ( 88.81-91.19 ) | ( 37.74-39.09 ) | ( 14.55-15.71 ) | ( 96.54-97.3) | (36.98, 37.01) |
| | K >= 5.5 + K < 6, K < 5 | | | | |
| Result | 61.21 | 68.12 | 27.45 | 89.91 | 69.77 |
| | ( 59.69-62.73 ) | ( 67.47-68.76 ) | ( 26.52-28.38 ) | ( 89.43-90.39 ) | (69.76-69.78) |
| SE = SP | 64.43 | 64.42 | 26.3 | 90.19 | 56.01 |
| | ( 62.94-65.93 ) | ( 63.76-65.08 ) | ( 25.43-27.18 ) | ( 89.7-90.67 ) | (55.99, 56.01) |
| 90% SE | 90.03 | 30.62 | 20.37 | 93.97 | 29.31 |
| | ( 89.1-90.97 ) | ( 29.99-31.25 ) | ( 19.77-20.97 ) | ( 93.39-94.55 ) | (29.29, 29.32) |
| | K >= 5 + K < 5.5, K < 5 | | | | |
| Result | 52.86 | 68.12 | 33.66 | 82.52 | 64.84 |
| | ( 51.61-54.11 ) | ( 67.47-68.76 ) | ( 32.71-34.59 ) | ( 81.94-83.1) | (64.83, 64.85) |
| SE = SP | 60.75 | 60.75 | 32.14 | 83.49 | 51.06 |
| | ( 59.53-61.98 ) | ( 60.07-61.43 ) | ( 31.29-32.99 ) | ( 82.89-84.1) | (51.05, 51.07) |
| 90% SE | 90.02 | 23.42 | 26.46 | 88.46 | 22.40 |
| | ( 89.27-90.77 ) | ( 22.84-24.01 ) | ( 25.86-27.05 ) | ( 87.6-89.32 ) | (22.38, 22.40) |

Utilizing the operating point characterized by equal sensitivity and specificity, the performance of the DNN in predicting hyperkalemia through ECG using lead-1 is as follows for each respective subset. When the serum potassium level greater than 7.5, the sensitivity and specificity were 80.95% (95% CI, 75.89-86.02%) and 80.95% (95% CI, 80.41-81.49%), respectively. Similarly, for cases within the serum potassium range of 6.5 to 7.5, the sensitivity and specificity exhibited values of 71.72% (95% CI, 69.45-73.99%) and 71.64% (95% CI, 71.02-72.26%).

As the serum potassium range narrowed to 6 to 6.5, the algorithm maintained a sensitivity of 67.95% (95% CI, 66.10-69.8%) and a specificity of 67.92% (95% CI, 67.27-68.57%). Progressing further, within the serum potassium range of 5.5 to 6, the algorithm demonstrated a sensitivity of 64.43% (95% CI, 62.94-65.93%) alongside a specificity of 64.42% (95% CI, 63.76-65.08%). Lastly, for serum potassium levels in the range of 5 to 5.5, the algorithm exhibited a sensitivity of 60.75% (95% CI, 59.53-61.98%) and a specificity of 60.75% (95% CI, 60.07-61.43%).

The operational point characterized by high sensitivity is indicative of an output suitable for a screening tool. When the serum potassium level exceeds 7.5, the algorithm demonstrated a sensitivity of 90% (95% CI, 86.18-93.98) alongside a specificity of 67.86% (95% CI 67.21-68.51). Similarly, when serum potassium range of 6.5 to 7.5, the sensitivity and specificity attained values of 90% (95% CI, 88.54-91.55%) and 45.80% (95% CI, 45.11-46.5%).

As the serum potassium range narrows to 6 to 6.5, the algorithm consistently upheld a sensitivity of 90% (95% CI, 88.81-91.19%) accompanied by a specificity of 38.42% (95% CI, 37.74-39.09). Progressing further, within the serum potassium range of 5.5 to 6, the algorithm showcased a sensitivity of 90% (95% CI, 89.1-90.97%) along with a specificity of 30.62% (95% CI, 29.99-31.25). Lastly, for serum potassium levels spanning from 5 to 5.5, the algorithm exhibited a sensitivity of 90% (95% CI, 89.27-90.77%) paired with a specificity of 23.42% (95% CI, 22.84-24.01)

The number of false-positive, false-negative, true-positive, and true-negative results for each model, as well as accuracy, is presented in Table 10. Table 10 is the confusion matrix for classification of hyperkalemia for different K values.

TABLE 10

| | TP | TN | FP | FN | Accuracy |
|---|---|---|---|---|---|
| | K > 7.5, K < 5 | | | | |
| Result | 207 | 13623 | 6377 | 24 | 68.36 |
| SE = SP | 187 | 16190 | 3810 | 44 | 80.95 |
| 90% SE | 208 | 13572 | 6428 | 23 | 68.11 |
| | K >= 6.5 + K <= 7.5, K < 5 | | | | |
| Result | 1148 | 13623 | 6377 | 369 | 68.65 |
| SE = SP | 1088 | 14328 | 5672 | 429 | 71.65 |
| 90% SE | 1366 | 9161 | 10839 | 151 | 48.92 |
| | K >= 6 + K < 6.5, K < 5 | | | | |
| Result | 1650 | 13623 | 6377 | 790 | 68.06 |
| SE = SP | 1658 | 13584 | 6416 | 782 | 67.92 |
| 90% SE | 2196 | 7683 | 12317 | 244 | 44.02 |
| | K >= 5.5 + K < 6, K < 5 | | | | |
| Result | 2413 | 13623 | 6377 | 1592 | 66.81 |
| SE = SP | 2540 | 12884 | 7116 | 1402 | 64.42 |
| 90% SE | 3549 | 6125 | 13875 | 393 | 40.41 |
| | K >= 5 + K < 5.5, K < 5 | | | | |
| Result | 3235 | 13623 | 6377 | 2885 | 64.54 |
| SE = SP | 3718 | 12150 | 7850 | 2402 | 60.75 |
| 90% SE | 5509 | 4685 | 15315 | 611 | 39.03 |

Collectively, the results indicate that the model demonstrates substantial accuracy in identifying hyperkalemia across populations, including populations afflicted with acute kidney disease (AKD) (Example 4) and chronic kidney disease (CKD) (Example 5), with performance varying according to serum potassium levels. Based on the AUC values across varying serum potassium levels, we can conclude that the algorithm performs well in identifying hyperkalemia, particularly in patients with critically high potassium levels. As serum potassium levels increase, the algorithm's accuracy (AUC) improves, reaching its peak at levels above 7.5 mmol/L.

While this invention is satisfied by configurations in many different forms, as described in detail in connection with preferred configurations of the invention, it is understood that the present disclosure is to be considered as exemplary of the principles of the invention and is not intended to limit the invention to the specific configurations illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, 16.

What is claimed is:

1. A process for screening for hyperkalemia from an electrocardiogram (ECG) signal, comprising:
segmenting a set of electrical data from a Lead I electrocardiogram (lead I ECG) signal into a plurality of segments, thereby providing a set of segmented Lead I ECG electrical data;
normalizing a plurality of waveforms in the set of segmented Lead I ECG data by:
i) applying a consistent time-frame to the plurality of waveforms;
ii) applying a consistent amplitude scale to the plurality of waveforms;
identifying the normalized waveform by a machine learning model trained to screen the normalized waveform for identifying a hyperkalemia waveform or a non-hyperkalemia waveform;
outputting a result of the screen.

2. The process of claim 1, wherein the consistent time-frame is scaled down to a plurality of datapoints.

3. The process of claim 2, wherein the plurality of datapoints are 962,434 parameters.

4. The process of claim 3, wherein the plurality of datapoints are no more than 1591 voltage measurements for each 5 second segment time-frame.

5. The process of claim 1, wherein the consistent amplitude scale is normalized from a global maximum and a global minimum.

6. The process of claim 1, wherein the model is trained to provide a binary classification of the normalized waveform as a hyperkalemia waveform or a non-hyperkalemia waveform.

7. The process of claim 6, wherein the model is instructed to apply a binary cross-entropy training metric, an Adam optimizer as a training metric, and accuracy as a training metric.

8. The process of claim 1, wherein the model is trained on at least 1,000 subjects.

9. The process of claim 1, wherein the model is a ResNet model.

10. The process of claim 1, wherein the model is trained to provide a tiered classification of the normalized waveform as a mild hyperkalemia waveform, moderate hyperkalemia waveform, severe hyperkalemia waveform, or a non-hyperkalemia waveform.

11. A system configured to execute the steps of a process of claim 1, operatively linked to an electrocardiogram (ECG) apparatus.

12. A system configured to execute the steps of a process of claim 1, operatively linked to an wrist apparatus configured for detecting a wrist-pulse Lead I ECG signal.

13. A system configured to execute the steps of a process of claim 1, operatively linked to one or more databases of electronic medical records or clinical data, or both.

14. The system of claim 1, wherein the electrocardiogram (ECG) signal is from a subject afflicted with acute kidney disease.

15. The system of claim 1, wherein the electrocardiogram (ECG) signal is from a subject afflicted with chronic kidney disease.

16. A process for training a model for detecting hyperkalemia from an electrocardiogram (ECG) signal, comprising:
inputting into a machine learning model a set of data from a database comprising a plurality of lead I electrocardiogram (lead I ECG) signals, whereby the set of data is sub-divided into two or more bins, whereby each bin is associated with a range of a serum potassium level from a subject, the set of data comprising at least two bins selected from:
a first bin comprising a first subset of Lead I electrocardiogram signals indicative of a subject having a potassium level >7.5 mEq/L;
a second bin comprising a second subset of Lead I electrocardiogram signals indicative of a subject having a potassium level ≥6.5 mEq/L and ≤7.5 mEq/L;
a third bin comprising a third subset of Lead I electrocardiogram signals indicative of a subject having a potassium level ≥6.0 mEq/L and <6.5 mEq/L;
a forth bin comprising a forth subset of Lead I electrocardiogram signals indicative of a subject having a potassium level ≥5.5 mEq/L and <6.0 mEq/L;
a fifth bin comprising a fifth subset of Lead I electrocardiogram signals indicative of a subject having a potassium level ≥5.0 mEq/L and <5.5 mEq/L;
a six bin whereby the normalized waveform corresponds to a normalized waveform indicative of a subject having a potassium level <5.0 mEq/L;
segmenting the set from the at least two bins into a plurality of segments, thereby providing a set of segmented lead I ECG data;
normalizing a plurality of waveforms in the set of segmented lead I ECG data by:
iii) applying a consistent time-frame to the plurality of waveforms;
iv) applying a consistent amplitude scale to the plurality of waveforms;
instructing a machine learning model to identify a pattern in a hyperkalemia normalized waveform or a pattern in a non-hyperkalemia normalized waveform based on a sensitivity threshold or a specificity threshold.

17. The process of claim 16, wherein the consistent time-frame is scaled down to a plurality of datapoints.

18. The process of claim 17, wherein the plurality of datapoints are 962,434 parameters.

19. The process of claim 18, wherein the plurality of datapoints are no more than 1591 voltage measurements for each 5 second segment time-frame.

20. The process of claim 16, wherein the consistent amplitude scale is normalized from a global maximum and a global minimum.

21. The process of claim 16, wherein the model is trained to provide a binary classification of the normalized waveform as a hyperkalemia waveform or a non-hyperkalemia waveform.

22. The process of claim 21, wherein the model has a sensitivity of about 90.0% or greater when providing a binary classification of hyperkalemia.

23. The process of claim 21, wherein the model has a specificity of about 90.0% or greater when providing a binary classification of hyperkalemia.

24. The process of claim 21, wherein the model is instructed to apply a binary cross-entropy training metric, an Adam optimizer as a training metric, and accuracy as a training metric.

25. The process of claim 16, wherein the model is trained on at least 1,000 subjects.

26. The process of claim 16, wherein the model is a ResNet model.

27. The process of claim 16, wherein the model is trained to provide a tiered classification of the normalized waveform as a mild hyperkalemia waveform, moderate hyperkalemia waveform, severe hyperkalemia waveform, or a non-hyperkalemia waveform.

28. The process of claim 16, wherein the lead I ECG signals are from a subject afflicted with acute kidney disease.

29. The process of claim 16, wherein the lead I ECG signals are from a subject afflicted with chronic kidney disease.
